(19) United States
(12) Patent Application Publication
(10) Pub. No.: US 2023/0124977 A1
(43) Pub. Date: Apr. 20, 2023

(54) CROSS-REACTIVE ANTIBODIES AND USES THEREOF

(71) Applicant: VANDERBILT UNIVERSITY, Nashville, TN (US)

(72) Inventors: Ivelin Stefanov Georgiev, Nashville, TN (US); Kelsey A. Pilewski, Nashville, TN (US)

(21) Appl. No.: 17/820,418

(22) Filed: Aug. 17, 2022

Related U.S. Application Data
(60) Provisional application No. 63/233,906, filed on Aug. 17, 2021.

(51) Int. Cl. C07K 16/10; C07K 16/12; A61P 31/04; A61P 31/14; A61P 31/18

(52) U.S. Cl. CPC C07K 16/109; C07K 16/10; C07K 16/1018; C07K 16/1045; C07K 16/1232; A61P 31/04; A61P 31/14; A61P 31/18; C07K 2317/31

(57) ABSTRACT

The present disclosure relates to broadly neutralizing antibodies and uses thereof for treating a pathogen infection or a co-infection of multiple pathogens.

### CROSS REFERENCE TO RELATED APPLICATIONS

This application claims the benefit of U.S. Provisional Patent Application Ser. No. 63/233,906 filed Aug. 17, 2021, the disclosure of which is expressly incorporated herein by reference in its entirety.

### STATEMENT REGARDING FEDERALLY SPONSORED RESEARCH

This invention was made with government support under Grant No. R01 AI131722 and R01 AI152693 awarded by the National Institutes of Health. The government has certain rights in the invention.

### REFERENCE TO A SEQUENCE LISTING

The Sequence Listing submitted Aug. 17, 2022 as an XML file named “10644-134US1_Sequence_Listing.xml,” created on Aug. 15, 2022, and having a size of 17,299,382 bytes is hereby incorporated by reference pursuant to 37 C.F.R. § 1.834.

### FIELD

The present disclosure relates to antibodies and uses thereof for treating infection.

### BACKGROUND

SARS-CoV-2, or the 2019 novel coronavirus (COVID-19), is a significant pandemic threat that has resulted in over 203,295,000 diagnosed cases including 4,303,515 deaths as of Aug. 10, 2021. The development of preventive and therapeutic measures that can counteract the ongoing, and any future, coronavirus pandemics is therefore of utmost significance for public health worldwide. Cross-reactivity between antigens occurs when an antibody directed against one specific antigen is successful in binding with another, different antigen. What is needed are novel cross-reactive antibodies and methods for treating SARS-CoV-2 infection or co-infection of SARS-CoV-2 and other pathogens.

### SUMMARY

In some aspects, disclosed herein is a method of treating a coronavirus infection in a subject in need comprising administering to the subject a therapeutically effective amount of a recombinant antibody, wherein the recombinant antibody comprises a light chain variable region (VL) that comprises a light chain complementarity determining region (CDRL)3 and a heavy chain variable region (VH) that comprises a heavy chain complementarity determining region (CDRH)3, wherein the CDRL3 comprises an amino acid sequence at least 60% identical to CQQSYNVPTF (SEQ ID NO: 1884), and wherein the CDRH3 comprises an amino acid sequence at least 60% identical to CAKGLTTESRLEFW (SEQ ID NO: 1818). In some embodiments, the CDRL3 comprises an amino acid sequence at least 95% identical to CQQSYNVPTF (SEQ ID NO: 1884). In some embodiments, the CDRH3 comprises an amino acid sequence at least 95% identical to CAKGLTTESRLEFW (SEQ ID NO: 1818). In some embodiments, the coronavirus comprises MERS-CoV, SARS-CoV, or SARS-CoV-2. In some embodiments, the subject is co-infected by a pathogen. In some embodiments, the virus is selected from the group consisting of influenza A, influenza B, HIV, and HCV. In some embodiments, the bacterium is Escherichia coli.

In some aspects, disclosed herein is a method of treating an influenza infection in a subject in need comprising administering to the subject a therapeutically effective amount of a recombinant antibody, wherein the recombinant antibody comprises a light chain variable region (VL) that comprises a light chain complementarity determining region (CDRL)3 and a heavy chain variable region (VH) that comprises a heavy chain complementarity determining region (CDRH)3, wherein the CDRL3 comprises an amino acid sequence at least 60% identical to CQQSYNVPTF (SEQ ID NO: 1884), and wherein the CDRH3 comprises an amino acid sequence at least 60% identical to CAKGLTTESRLEFW (SEQ ID NO: 1818). In some embodiments, the influenza is influenza A or influenza B. In some embodiments, the subject is co-infected by a pathogen. In some embodiments, the pathogen is a virus or a bacterium. In some embodiments, the virus is selected from the group consisting of MERS-CoV, SARS-CoV, SARS-CoV-2, HIV, and HCV. In some embodiments, the bacterium is Escherichia coli.

In some aspects, disclosed herein is a method of treating an Escherichia coli infection in a subject in need comprising administering to the subject a therapeutically effective amount of a recombinant antibody, wherein the recombinant antibody comprises a light chain variable region (VL) that comprises a light chain complementarity determining region (CDRL)3 and a heavy chain variable region (VH) that comprises a heavy chain complementarity determining region (CDRH)3, wherein the CDRL3 comprises an amino acid sequence at least 60% identical to CQQSYNVPTF (SEQ ID NO: 1884), and wherein the CDRH3 comprises an amino acid sequence at least 60% identical to CAKGLTTESRLEFW (SEQ ID NO: 1818). In some embodiments, the subject is co-infected by a pathogen. In some embodiments, the pathogen is a virus or a bacterium. In some embodiments, the virus is selected from the group consisting of MERS-CoV, SARS-CoV, SARS-CoV-2, influenza A, influenza B, HIV, and HCV.

In some aspects, disclosed herein is a method of treating a coronavirus infection in a subject in need comprising administering to the subject a therapeutically effective amount of a recombinant antibody, wherein the recombinant antibody comprises a light chain variable region (VL) that comprises a light chain complementarity determining region (CDRL)1, a CDRL2, and a CDRL3 and a heavy chain variable region (VH) that comprises a heavy chain complementarity determining region (CDRH)1, CDRH2, CDRH3, wherein
CDRL1 is SEQ ID NO: 13121,
CDRL2 is SEQ ID NO: 13127,
CDRL3 is SEQ ID NO: 25 or 1884,
CDRH1 is SEQ ID NO: 13103,
CDRH2 is SEQ ID NO: 13109, and
CDRH3 is SEQ ID NO: 1818.

In some aspects, disclosed herein is a method of treating an influenza infection in a subject in need comprising administering to the subject a therapeutically effective amount of a recombinant antibody, wherein the recombinant antibody comprises a light chain variable region (VL) that comprises a light chain complementarity determining region (CDRL)1, a CDRL2, and a CDRL3 and a heavy chain variable region (VH) that comprises a heavy chain complementarity determining region (CDRH)1, CDRH2, CDRH3, wherein
CDRL1 is SEQ ID NO: 13121,
CDRL2 is SEQ ID NO: 13127,
CDRL3 is SEQ ID NO: 25 or 1884,
CDRH1 is SEQ ID NO: 13103,
CDRH2 is SEQ ID NO: 13109, and
CDRH3 is SEQ ID NO: 1818.

In some aspects, disclosed herein is a method of treating an Escherichia coli infection in a subject in need comprising administering to the subject a therapeutically effective amount of a recombinant antibody, wherein the recombinant antibody comprises a light chain variable region (VL) that comprises a light chain complementarity determining region (CDRL)1, a CDRL2, and a CDRL3 and a heavy chain variable region (VH) that comprises a heavy chain complementarity determining region (CDRH)1, CDRH2, CDRH3, wherein
CDRL1 is SEQ ID NO: 13121,
CDRL2 is SEQ ID NO: 13127,
CDRL3 is SEQ ID NO: 25 or 1884,
CDRH1 is SEQ ID NO: 13103,
CDRH2 is SEQ ID NO: 13109, and
CDRH3 is SEQ ID NO: 1818.

### BRIEF DESCRIPTION OF DRAWINGS

The accompanying figures, which are incorporated in and constitute a part of this specification, illustrate aspects described below.

FIGS. 1A-1G show discovery of Exceptionally Broad Anti-viral Antibodies from a Chronically HIV-1/HCV Co-infected Donor. FIG. 1A shows plot of maximum LIBRA-seq score for an HIV antigen (Ag) vs. HCV antigen where each dot represents a single class-switched (IgG, IgA) cell. Colored dots represent antibodies selected for further study. Cells with antigen values <0 were set to −4. FIG. 1B shows genetic features of identified HIV-1/HCV cross-reactive BCRs. Color of antibody name corresponds to LIBRA-seq score colors in A. FIG. 1C shows binding of HIV/HCV cross-reactive recombinant antibodies to the panel of viral antigens (shown on the left) at 10 ug/mL as measured by ELISA. The following antibodies are shown as controls: AR3C (HCV E2), VRC01 (HIV-1 gp120). FIGS. 1D-1E show recombinantly-expressed lead antibody binding to clade B (B41) and clade A (BG505) HIV envelope glycoproteins; and (FIGS. 1F-1G) genotype 2a (JFH1) and genotype 1a (H77) HCV envelope glycoproteins, measured by ELISA. The sequences in FIG. 1 include: CAKGLTTESRLEFW (SEQ ID NO: 1818), CVSSWGPESPYYFDYW (SEQ ID NO: 1819), CAREYCTGGDCHFFLDYW (SEQ ID NO: 1820), CARVAPPGVVNNKWFDIW (SEQ ID NO: 1821), CARSEKRVTMTRKIKGRWFGPW (SEQ ID NO: 1822), CQQSYNVPTF (SEQ ID NO: 1884), CHQSSSLPFTF (SEQ ID NO: 1885), CQHFYSSPPTF (SEQ ID NO: 1886), CCLYAGSYSWVF (SEQ ID NO: 1887), and CQVWDSSSEHVVF (SEQ ID NO: 1888).

FIGS. 2A-2F show that HIV-1/HCV cross-reactive antibodies recognize distinct epitopes on the HIV-1 and HCV Envelope Glycoproteins. (FIG. 2A) Antibody binding to the E1 (strain: HC-J4; left) and E2 (strain: JFH-1; right) subunits of the HCV envelope glycoprotein measured by ELISA. The HCV1 E2-specific antibody HCV1 is shown as a positive control, and the HIV-1-specific antibody VRC01 is shown as a negative control. (FIG. 2B) Antibody binding to HCV E2 glycoprotein (strain: JFH-1) in the presence of 1M D-(+)-Mannose. Data plotted as % inhibition as compared to binding in PBS buffer. Antibodies demonstrating negative % inhibition values were set to 0 (FIG. 2C) Antibody binding to cell surface-expressed HCV E1E2 (strain: H77) measured by flow cytometry. Cells were either pre-incubated with PBS (filled bar) or CD81-LEL (open bar) before detection by fluorescent secondary. Data shown as background-subtracted mean fluorescence intensity (MFI). (FIG. 2D) Antibody binding to the gp120 (strain AE.A244) (left) and gp41 (strain: MN) (right) subunits of the HIV-1 envelope glycoprotein measured by ELISA. The gp120-specific and gp41-specific antibodies VRC01 and 2F5, respectively, are shown as positive controls, and the HCV-specific antibody HCV1 is shown as a negative control. (FIG. 2E) Antibody binding to HIV-1 gp140 (strain: BG505) in the presence of 1M D-(+)-Mannose measured ELISA. Data plotted as % inhibition as compared to binding in PBS buffer. The mannose-independent HIV-1 antibody VRC01 is shown as a control. Antibodies demonstrating negative % inhibition values were set to 0. (FIG. 2F) Antibody binding to HIV-1 gp140 (strain: BG505) in the presence of competitor antibodies measured by ELISA. Competitor antibodies (shown on the Y axis) were added first, and subsequently binding of biotinylated analyte to antibodies (shown on the X axis) was detected. Data displayed as % inhibition, calculated as a function of no competition controls.

FIGS. 3A-3G show that HIV-1/HCV Cross-reactive antibodies show diverse neutralization and fc-mediated effector functions. FIG. 3A shows in vitro neutralization of a panel of diverse genotype 1 HCV virus strains by HIV-1/HCV-reactive antibodies and control antibody HEPC74 at 100 μg/mL. % Neutralization calculated in comparison to unrelated IgG control. FIG. 3B shows antibody-dependent cellular phagocytosis (ADCP) by either HIV-1/HCV cross-reactive or control antibodies (HCV1: positive; Palivizumab: negative) against HCV E1E2 envelope protein (H77). FIG. 3C shows area under the curve (AUC) values computed from (FIG. 3B). FIG. 3D shows antibody-dependent cellular cytotoxicity (ADCC) potentiated by either HIV-1/HCV cross-reactive or control antibodies (PGT151/HIVIG C: positive; Palivizumab: negative) against infectious HIV-1 envelope protein (CE1086). FIG. 3E shows area under the curve (AUC) values computed from (FIG. 3D). FIG. 3F shows antibody-dependent cellular phagocytosis (ADCP) by either HIV-1/HCV cross-reactive or control antibodies (CAP256.26.25/HIVIG C: positive; Palivizumab: negative) against HIV-1 envelope protein (BG505). FIG. 3G shows area under the curve (AUC) computed from (FIG. 3F).

FIGS. 4A-4E show that cross-reactive mab688 reveals exceptionally broad anti-viral functions achieved by glycan recognition. FIG. 4A shows binding of HIV-1/HCV cross-reactive antibodies (columns) to a panel of diverse viral antigens (rows) at 10 μg/mL, as measured by ELISA. The following antibodies are shown as controls: HCV1 (HCV E2), VRC01 (HIV-1 gp120), FE53 (HA), Palivizumab (RSV F), CR3022 (SARS-CoV/SARS-CoV-2 S), 1F8 (MERS S). FIG. 4B shows mAb688 binding to SARS-CoV-2 spike protein with PBS (left) and competition in presence of 1M D-(+)-Mannose (right), displayed as % inhibition (y-axis) in the presence of mannose. FIG. 4C shows mAb688 binding to Influenza A HA (strain H1N1/New Caledonia/1999) envelope glycoprotein with PBS (left) and competition in presence of 1M D-(+)-Mannose (right), displayed as % inhibition (y-axis) in the presence of mannose. FIG. 4D shows antibody-dependent cellular phagocytosis (ADCP) of SARS-CoV-2 envelope glycoprotein (left), with data displayed as area under the curve (AUC, right). CR3022 is shown as a positive control, and Palivizumab is shown as a negative control. FIG. 4E shows antibody-dependent cellular phagocytosis (ADCP) of Influenza A HA (strain H1N1/New Caledonia/1999) envelope glycoprotein (left), with data displayed as AUC (right). CR9114 is shown as a positive control, and Palivizumab is shown as a negative control.

FIGS. 5A-5D show that somatic hypermutation establishes and enhances cross-reactivity. Binding of germline-reverted antibody mutants (FIG. 5A) mAb180 and (FIG. 5B) mAb692 to HIV-1 gp140 (strain: BG505, gray bars) and HCV E2 (strain: JFH1, black bars) measured by ELISA. Data shown was calculated by dividing the area under the ELISA curve (AUC) of the germline-reverted antibody by the AUC of the native antibody (% Native binding). Binding of early sequences clonally related to (FIG. 5C) mAb180 or (FIG. 5D) mAb692 to HIV-1 gp140 (strain: BG505, left) or HCV E2 (strain: JFH1, right) measured by ELISA. Data is shown as a heatmap where % Native binding is calculated as in (FIGS. 5A-5B). Each heatmap square represents a unique combination of heavy and light chain sequences. The phylogenetic relationship between each set of sequences was determined using PhyML, not shown to scale.

FIGS. 6A-6C show strategy for the Identification of HIV-1/HCV cross-reactive B cells using LIBRA-seq. FIG. 6A shows diverse, DNA-barcoded envelope proteins from the indicated strains of both HIV-1 and HCV were used to isolate antigen-specific B cells from Vanderbilt HIV-1 cohort donor VC10014. FIG. 6B shows flow sorting strategy for the identification of antigen-specific B cells. First, the lymphocyte population was identified by scatter, followed by exclusion of dead cells, monocytes, and IgM-expressing cells. Next, the B cell population was identified, and Dead⁻, CD14⁻, IgM⁻, CD3⁻, CD19⁺, Antigen⁺ cells were sorted for subsequent single-cell sequencing. FIG. 6C shows frequency of B cells (y-axis) that were positive for each combination (filled circles connected by lines) of antigens (rows), colored by isotype. B cells with calculated LIBRA-seq scores >1 for a given antigen were considered positive.

FIGS. 7A-7G show characteristics of HCV and HIV-1 Envelope Recognition by HIV-1/HCV Antibodies. FIG. 7A shows antibody binding to PNGaseF de-glycosylated HCV E2 (strain: JFH1) measured by ELISA was compared to wild type (WT) binding. HCV-specific antibody AR3C is shown as a control. FIG. 7B shows antibody binding to HCV E2 (strain:JFH1) after mannosidase inhibitor treatment. AP33 is shown as a control. FIG. 7C shows nsEM images of HIV-1 gp140 in complex with either mAb180 (left: Fab, colored yellow) or mAb692 (right: Fab, colored yellow). FIG. 7D shows HIV-specific antibody binding to HCV envelope protein measured by ELISA. HCV-specific antibody AR3C is shown as a control. FIG. 7E shows antibody binding to PNGaseF de-glycosylated HIV-1 gp140 (strain: BG505) measured by ELISA was compared to wild type (WT) binding. HIV-specific antibody VRC01 is shown as a control. FIG. 7F shows antibody binding to HIV-1 Env (BG505) after mannosidase inhibitor treatment. FIG. 7G 7VRC01 is shown as a control.

FIGS. 8A-8C show HIV-1 and HCV Antibody Neutralization Phenotypes. FIG. 8A shows neutralization of HCV-pseudotyped viruses by HIV-1/HCV cross-reactive antibodies and human IgG control. For each antibody (panel) and concentration (x-axis) against each virus (colored curve), shown is % neutralization (y-axis) calculated as a function of no-antibody control. FIG. 8B shows half-maximal inhibitory concentration (IC50, μg/mL) of each antibody against the respective genotype 1 HCV virus strains from (FIG. 8A). ND=Not determined. FIG. 8C shows neutralization (IC50, μg/mL) of a panel of HIV-1-pseudotyped viruses by HIV-1/HCV cross-reactive antibodies.

FIGS. 9A-9G show mAb688 Functional Assays Against SARS-CoV-2, Influenza, and Escherichia coli. FIG. 9A shows mAb688 binding to WT or specific glycan-mutated SARS-CoV-2 S. FIG. 8B shows antibody-mediated neutralization of SARS-CoV-2-pseudotyped VSV particles. Antibody CoV2-2381 is shown as a positive control. FIG. 9C shows antibody inhibition of hemagglutination mediated by Influenza A HA (left: A/Brisbane/02/2018 (CA/09 pdm-like H1N1), right: A/Hong Kong/4801/2014 (H3N2)). Stem-specific antibodies CR6261 and CR8020 are shown as negative controls. Head-specific antibodies 1F8 and #585 are shown as positive controls. FIGS. 8D-8E show antibody-mediated neutralization of (FIG. 9D) Influenza A (IAV; A/California/07/2009 (pdm H1N1) or (FIG. 9E) Influenza B (IBV; B/Massachusetts/02/2012). Antibodies #7665, #1664, and #46 are shown as positive controls. FIG. 9F shows bacterial cell adherence to bladder epithelial cells was measured for both the WT UPEC strain UTI89 and bacteria lacking FimA-H, in the presence of either media, mannose or antibody. Bacterial adherence in the presence of 20 μg/mL mAb688 or the isotype control HCV1 is shown. Statistical significance determined by t-test. *** denotes p<0.001;**** denotes p<0.0001. FIG. 9G shows measurement of E. coli strain UTI89-mediated hemagglutination in the presence of either media, mannose or antibody. Hemagglutination in the presence of 20 μg/mL mAb688 or the isotype control HCV1 is shown. Statistical significance determined by one-way ANOVA with Dunnett's test for multiple comparisons. **** denotes p<0.0001.

FIG. 10 shows that mAb688 achieves broad anti-viral binding via recognition of immature glycans. Binding of mAb688 to a printed glycan microarray was tested at 50 μg/mL. Antibody binding was detected using fluorescent secondary antibody and data is shown as the average Relative Fluorescence Units (RFU). Glycan structures corresponding to numbered green circles are shown below.

FIGS. 11A-11C show autoreactivity of HIV-1/HCV cross-reactive antibodies. FIG. 11A shows antibody binding to a panel of autoantigens using the AtheNA multiplex assay, over a concentration gradient. Relative units (RU)>100 are considered positive. The HIV-1 antibody 4E10 is shown as a positive control, and mAb82 is shown as a negative control. FIGS. 11B-11C show antibody binding to whole, unpermeabilized, un-infected HEp-2 cells detected by anti-IgG-PE (anti-IgA-PE; mAb803, mAbKP1-8). FIG. 11B shows secondary only (negative) and 4E10 (positive) control plots. FIG. 11C shows binding of HIV/HCV cross-reactive antibodies depicted as MFI of the PE channel (-MFI unstained control) at the shown concentration of antibody.

FIGS. 12A-12C shows that native antibody features are crucial to binding viral envelopes. Binding of germline-reverted (FIG. 12A) mAb688 and/or isotype antibody mutants (FIG. 12B) mAb803, and (FIG. 12C) mAbKP1-8 to HIV-1 gp140 (strain: BG505, left) and HCV E1E2 (strain: H77, right) by ELISA.

FIGS. 13A-13B show cross-reactive antibody binding phenotypes in varying buffer conditions. Binding to either HCV (FIG. 13A) or HIV-1 (FIG. 13B) envelope glycoproteins by ELISA in the presence of the buffer conditions shown. Antigens were diluted in PBS, pH=7.4 and captured on ELISA plates coated with 500 ng/well GNA (Snowdrop) lectin, and buffer conditions shown were used for blocking, primary, and secondary antibody dilution steps, all buffers were prepared in PBS-T (PBS+0.05% Tween-20). Data shown was normalized to binding in the presence of PBS-T, pH=7.4 (WT binding).

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

In the present invention, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. Native antibodies are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 RI, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv, nanoantibody and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The terms “antigen binding site”, “binding site” and “binding domain” refer to the specific elements, parts or amino acid residues of a polypeptide, such as an antibody, that bind the antigenic determinant or epitope.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

The term “CDR” as used herein refers to the “complementarity determining regions” of the antibody which consist of the antigen binding loops. (Kabat E. A. et al., (1991) Sequences of proteins of immunological interest. NIH Publication 91-3242). Each of the two variable domains of an antibody Fv fragment contain, for example, three CDRs.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant antibody” refers to an amount of a recombinant antibody sufficient to prevent, treat, or mitigate a pathogen infection.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to an antigen, and/or ameliorating an infection.

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “nanobody”, “V_{H}H”, “V_{H}H antibody fragment” and “single domain antibody” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells trans-formed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “specificity” refers to the number of different types of antigens or antigenic determinants to which a particular antigen-binding molecule (such as the recombinant antibody of the invention) can bind. As used herein, the term “specifically binds,” as used herein with respect to a recombinant antibody refers to the recombinant antibody's preferential binding to one or more epitopes as compared with other epitopes. Specific binding can depend upon binding affinity and the stringency of the conditions under which the binding is conducted. In one example, an antibody specifically binds an epitope when there is high affinity binding under stringent conditions.

It should be understood that the specificity of an antigen-binding molecule (e.g., the recombinant antibodies of the present invention) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (KA), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as the recombinant antibodies of the present invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the recombinant antibodies of the invention) will bind to their antigen with a dissociation constant (K_{D}) of 10⁻⁵ to 10⁻¹² moles/liter or less, and preferably 10⁻⁷ to 10⁻¹² moles/liter or less, and more preferably 10⁻⁸ to 10⁻¹² moles/liter.

“Therapeutically effective amount” refers to the amount of a composition such as recombinant antibody that will elicit the biological or medical response of a tissue, system, animal, or human that is being sought by the researcher, veterinarian, medical doctor or other clinician over a generalized period of time. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of recombinant antibodies as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased viral/bacterial titers, decreased viral RNA levels, and/or prolonged survival of a subject. It will be understood that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms of a cancer or condition and/or alleviating, mitigating or impeding one or more causes of a cancer. Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection, or during chronic infection. Prophylactic administration can occur for several minutes to months prior to the manifestation of an infection.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

### Antibodies and Methods

In some aspects, disclosed herein is method of treating a coronavirus infection in a subject in need comprising administering to the subject a therapeutically effective amount of a recombinant antibody, wherein the recombinant antibody comprises a light chain variable region (VL) that comprises a light chain complementarity determining region (CDRL)3 and/or a heavy chain variable region (VH) that comprises a heavy chain complementarity determining region (CDRH)3, wherein the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to CQQSYNVPTF (SEQ ID NO: 1884), and wherein the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1818)
CAKGLTTESRLEFW.

In some embodiments, the CDRL3 comprises an amino acid sequence at least 60% (for example, at least 60%, at least 65%, at least 70%, at least 75%, at least 80%, at least 85%, at least 90%, at least 95%, at least 96%, or at least 97%, at least 98%, at least 99%) identical to

(SEQ ID NO: 1888)
CQVWDSSSEHVVF;
(SEQ ID NO: 1887)
CCLYAGSYSWVF;
(SEQ ID NO: 1883)
CMQPLQLPDTF;
(SEQ ID NO: 1884)
CQQSYNVPTF;
(SEQ ID NO: 1885)
CHQSSSLPFTF;
or
(SEQ ID NO: 1886)
CQHFYSSPPTF

and/or

the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1822)
CARSEKRVTMTRKIKGRWFGPW;
(SEQ ID NO: 1821)
CARVAPPGVVNNKWFDIW;
(SEQ ID NO: 1817)
CAAGLWSGDLSRPRYSDSW;
(SEQ ID NO: 1818)
CAKGLTTESRLEFW;
(SEQ ID NO: 1819)
CVSSWGPESPYYFDYW;
(SEQ ID NO: 1820)
CAREYCTGGDCHFFLDYW;
or
(SEQ ID NO: 101)
QVWDSSSEHVV.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13102,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13108,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1817,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13120,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13126, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13103,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13109,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1818,
CDRL1 comprises an amino acid sequence at least 60% (for example, at least 60%, at least 65%, at least 70%, at least 75%, at least 80%, at least 85%, or at least 90%, at least 95%, at least 96%, at least 97%, at least 98%, or at least 99%) identical to SEQ ID NO: 13121,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13127, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13104,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13110,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1819,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13122,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13108, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13105,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13111,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1820,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13123,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13129, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13106,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13112,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 104 or 1821,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13124,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13130, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13107.
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13113,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 110 or 1822,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13125,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13131, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 101 or 1888.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13102,
CDRH2 is SEQ ID NO: 13108,
CDRH3 is SEQ ID NO: 1817,
CDRL1 is SEQ ID NO: 13120,
CDRL2 is SEQ ID NO: 13126, and
CDRL3 is SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, CDRH3, wherein:
CDRH1 is SEQ ID NO: 13103,
CDRH2 is SEQ ID NO: 13109,
CDRH3 is SEQ ID NO: 1818,
CDRL1 is SEQ ID NO: 13121,
CDRL2 is SEQ ID NO: 13127, and
CDRL3 is SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13104,
CDRH2 is SEQ ID NO: 13110,
CDRH3 is SEQ ID NO: 1819,
CDRL1 is SEQ ID NO: 13122,
CDRL2 is SEQ ID NO: 13128, and
CDRL3 is SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13105,
CDRH2 is SEQ ID NO: 13111,
CDRH3 is SEQ ID NO: 1820,
CDRL1 is SEQ ID NO: 13123,
CDRL2 is SEQ ID NO: 13129, and
CDRL3 is SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13106,
CDRH2 is SEQ ID NO: 13112,
CDRH3 is SEQ ID NO: 104 or 1821,
CDRL1 is SEQ ID NO: 13124,
CDRL2 is SEQ ID NO: 13130, and
CDRL3 is SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13107,
CDRH2 is SEQ ID NO: 13113,
CDRH3 is SEQ ID NO: 110 or 1822,
CDRL1 is SEQ ID NO: 13125,
CDRL2 is SEQ ID NO: 13131, and
CDRL3 is SEQ ID NO: 101 or 1888.

In some embodiments, the method comprises administering to the subject the antibody disclosed in International Application Publication No. WO2020/206232, which is incorporated herein by reference in its entirety.

In some embodiments, the coronavirus comprises avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV. In some embodiments, the coronavirus comprises MERS-CoV, SARS-CoV, or SARS-CoV-2.

In some embodiments, the subject is co-infected by a pathogen. In some embodiments, the virus is selected from the group consisting of influenza A, influenza B, HIV, and HCV. In some embodiments, the bacterium is Escherichia coli.

In some aspects, disclosed herein is method of treating an influenza infection in a subject in need comprising administering to the subject a therapeutically effective amount of a recombinant antibody, wherein the recombinant antibody comprises a light chain variable region (VL) that comprises a light chain complementarity determining region (CDRL)3 and/or a heavy chain variable region (VH) that comprises a heavy chain complementarity determining region (CDRH)3, wherein the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to CQQSYNVPTF (SEQ ID NO: 1884), and wherein the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1818)
CAKGLTTESRLEFW.

In some embodiments, the CDRL3 comprises an amino acid sequence at least 60% (for example, at least 60%, at least 65%, at least 70%, at least 75%, at least 80%, at least 85%, at least 90%, at least 95%, at least 96%, or at least 97%, at least 98%, at least 99%) identical to

(SEQ ID NO: 1888)
CQVWDSSSEHVVF;
(SEQ ID NO: 1887)
CCLYAGSYSWVF;
(SEQ ID NO: 1883)
CMQPLQLPDTF;
(SEQ ID NO: 1884)
CQQSYNVPTF;
(SEQ ID NO: 1885)
CHQSSSLPFTF;
or
(SEQ ID NO: 1886)
CQHFYSSPPTF

and/or

the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1822)
CARSEKRVTMTRKIKGRWFGPW;
(SEQ ID NO: 1821)
CARVAPPGVVNNKWFDIW;
(SEQ ID NO: 1817)
CAAGLWSGDLSRPRYSDSW;
(SEQ ID NO: 1818)
CAKGLTTESRLEFW;
(SEQ ID NO: 1819)
CVSSWGPESPYYFDYW;
(SEQ ID NO: 1820)
CAREYCTGGDCHFFLDYW;
or
(SEQ ID NO: 101)
QVWDSSSEHVV.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13102,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13108,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1817,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13120,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13126, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13103,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13109,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1818,
CDRL1 comprises an amino acid sequence at least 60% (for example, at least 60%, at least 65%, at least 70%, at least 75%, at least 80%, at least 85%, or at least 90%, at least 95%, at least 96%, at least 97%, at least 98%, or at least 99%) identical to SEQ ID NO: 13121,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13127, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13104,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13110,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1819,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13122,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13108, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13105,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13111,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1820,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13123,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13129, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13106,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13112,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 104 or 1821,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13124,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13130, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13107,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13113,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 110 or 1822,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13125,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13131, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 101 or 1888.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13102,
CDRH2 is SEQ ID NO: 13108,
CDRH3 is SEQ ID NO: 1817,
CDRL1 is SEQ ID NO: 13120,
CDRL2 is SEQ ID NO: 13126, and
CDRL3 is SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, CDRH3, wherein:
CDRH1 is SEQ ID NO: 13103,
CDRH2 is SEQ ID NO: 13109,
CDRH3 is SEQ ID NO: 1818,
CDRL1 is SEQ ID NO: 13121,
CDRL2 is SEQ ID NO: 13127, and
CDRL3 is SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13104,
CDRH2 is SEQ ID NO: 13110,
CDRH3 is SEQ ID NO: 1819,
CDRL1 is SEQ ID NO: 13122,
CDRL2 is SEQ ID NO: 13128, and
CDRL3 is SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13105,
CDRH2 is SEQ ID NO: 13111,
CDRH3 is SEQ ID NO: 1820,
CDRL1 is SEQ ID NO: 13123,
CDRL2 is SEQ ID NO: 13129, and
CDRL3 is SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13106,
CDRH2 is SEQ ID NO: 13112,
CDRH3 is SEQ ID NO: 104 or 1821,
CDRL1 is SEQ ID NO: 13124,
CDRL2 is SEQ ID NO: 13130, and
CDRL3 is SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13107,
CDRH2 is SEQ ID NO: 13113,
CDRH3 is SEQ ID NO: 110 or 1822,
CDRL1 is SEQ ID NO: 13125,
CDRL2 is SEQ ID NO: 13131, and
CDRL3 is SEQ ID NO: 101 or 1888.

In some embodiments, the method comprises administering to the subject an antibody disclosed in WO2020/206232, which is incorporated herein by reference in its entirety.

In some embodiments, the influenza is influenza A or influenza B. In some embodiments, the subject is co-infected by a pathogen. In some embodiments, the pathogen is a virus or a bacterium. In some embodiments, the virus is selected from the group consisting of a coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), HIV, and HCV. In some embodiments, the bacterium is Escherichia coli.

In some aspects, disclosed herein is method of treating an Escherichia coli infection in a subject in need comprising administering to the subject a therapeutically effective amount of a recombinant antibody, wherein the recombinant antibody comprises a light chain variable region (VL) that comprises a light chain complementarity determining region (CDRL)3 and/or a heavy chain variable region (VH) that comprises a heavy chain complementarity determining region (CDRH)3, wherein the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to CQQSYNVPTF (SEQ ID NO: 1884), and wherein the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1818)
CAKGLTTESRLEFW.

In some embodiments, the CDRL3 comprises an amino acid sequence at least 60% (for example, at least 60%, at least 65%, at least 70%, at least 75%, at least 80%, at least 85%, at least 90%, at least 95%, at least 96%, or at least 97%, at least 98%, at least 99%) identical to

(SEQ ID NO: 1888)
CQVWDSSSEHVVF;
(SEQ ID NO: 1887)
CCLYAGSYSWVF;
(SEQ ID NO: 1883)
CMQPLQLPDTF;
(SEQ ID NO: 1884)
CQQSYNVPTF;
(SEQ ID NO: 1885)
CHQSSSLPFTF;
or
(SEQ ID NO: 1886)
CQHFYSSPPTF

and/or

the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1822)
CARSEKRVTMTRKIKGRWFGPW;
(SEQ ID NO: 1821)
CARVAPPGVVNNKWFDIW;
(SEQ ID NO: 1817)
CAAGLWSGDLSRPRYSDSW;
(SEQ ID NO: 1818)
CAKGLTTESRLEFW;
(SEQ ID NO: 1819)
CVSSWGPESPYYFDYW;
(SEQ ID NO: 1820)
CAREYCTGGDCHFFLDYW;
or
(SEQ ID NO: 101)
QVWDSSSEHVV.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13102,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13108,
CDRH3 comprises an amino acid sequence at least 60% (for example, at least 60%, at least 65%, at least 70%, at least 75%, at least 80%, at least 85%, or at least 90%, at least 95%, at least 96%, at least 97%, at least 98%, or at least 99%) identical to SEQ ID NO: 1817,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13120,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13126, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13103,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13109,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1818,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13121,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13127, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13104,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13110,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1819,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13122,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13128, and/or
CDRL3 comprises an amino acid sequence at least 60% (for example, at least 60%, at least 65%, at least 70%, at least 75%, at least 80%, at least 85%, or at least 90%, at least 95%, at least 96%, at least 97%, at least 98%, or at least 99%) identical to SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13105,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13111,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1820,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13123,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13129, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13106,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13112,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 104 or 1821,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13124,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13130, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13107,
CDRH2 comprises an amino acid sequence at least 60% (for example, at least 60%, at least 65%, at least 70%, at least 75%, at least 80%, at least 85%, or at least 90%, at least 95%, at least 96%, at least 97%, at least 98%, or at least 99%) identical to SEQ ID NO: 13113,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 110 or 1822,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13125,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13131, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 101 or 1888.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13102,
CDRH2 is SEQ ID NO: 13108,
CDRH3 is SEQ ID NO: 1817,
CDRL1 is SEQ ID NO: 13120,
CDRL2 is SEQ ID NO: 13126, and
CDRL3 is SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13103,
CDRH2 is SEQ ID NO: 13109,
CDRH3 is SEQ ID NO: 1818,
CDRL1 is SEQ ID NO: 13121,
CDRL2 is SEQ ID NO: 13127, and
CDRL3 is SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13104,
CDRH2 is SEQ ID NO: 13110,
CDRH3 is SEQ ID NO: 1819,
CDRL1 is SEQ ID NO: 13122,
CDRL2 is SEQ ID NO: 13128, and
CDRL3 is SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13105,
CDRH2 is SEQ ID NO: 13111,
CDRH3 is SEQ ID NO: 1820,
CDRL1 is SEQ ID NO: 13123,
CDRL2 is SEQ ID NO: 13129, and
CDRL3 is SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13106,
CDRH2 is SEQ ID NO: 13112,
CDRH3 is SEQ ID NO: 104 or 1821,
CDRL1 is SEQ ID NO: 13124,
CDRL2 is SEQ ID NO: 13130, and
CDRL3 is SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13107,
CDRH2 is SEQ ID NO: 13113,
CDRH3 is SEQ ID NO: 110 or 1822,
CDRL1 is SEQ ID NO: 13125,
CDRL2 is SEQ ID NO: 13131, and
CDRL3 is SEQ ID NO: 101 or 1888.

In some embodiments, the method comprises administering to the subject the antibody disclosed in International Application Publication No. WO2020/206232, which is incorporated herein by reference in its entirety.

In some embodiments, the subject is co-infected by a pathogen. In some embodiments, the pathogen is a virus or a bacterium. In some embodiments, the virus is selected from the group consisting of a coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), influenza A, influenza B, HIV, and HCV.

In some aspects, disclosed herein is a method for treating, reducing, decreasing, inhibiting, and/or preventing an infectious disease in a subject comprising administering to the subject a therapeutically effective amount of any of the antibodies disclosed herein. In some embodiments, the subject is infected by more than one pathogen. In some embodiments, the infectious disease is caused by infection of a virus, such as, for example, an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In some embodiments, the infectious disease is caused by a bacterial infection, wherein the bacterial infection is an infection with a bacterium selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

In some embodiments, the method comprises administering to the subject a therapeutically effective amount of a recombinant antibody, wherein the recombinant antibody comprises a light chain variable region (VL) that comprises a light chain complementarity determining region (CDRL)3 and/or a heavy chain variable region (VH) that comprises a heavy chain complementarity determining region (CDRH)3, wherein the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to CQQSYNVPTF (SEQ ID NO: 1884), and wherein the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to CAKGLTTESRLEFW (SEQ ID NO: 1818).

In some embodiments, the CDRL3 comprises an amino acid sequence at least 60% (for example, at least 60%, at least 65%, at least 70%, at least 75%, at least 80%, at least 85%, at least 90%, at least 95%, at least 96%, or at least 97%, at least 98%, at least 99%) identical to

(SEQ ID NO: 1888)
CQVWDSSSEHVVF;
(SEQ ID NO: 1887)
CCLYAGSYSWVF;
(SEQ ID NO: 1883)
CMQPLQLPDTF;
(SEQ ID NO: 1884)
CQQSYNVPTF;
(SEQ ID NO: 1885)
CHQSSSLPFTF;
or
(SEQ ID NO: 1886)
CQHFYSSPPTF

and/or

the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to

(SEQ ID NO: 1822)
CARSEKRVTMTRKIKGRWFGPW;
(SEQ ID NO: 1821)
CARVAPPGVVNNKWFDIW;
(SEQ ID NO: 1817)
CAAGLWSGDLSRPRYSDSW;
(SEQ ID NO: 1818)
CAKGLTTESRLEFW;
(SEQ ID NO: 1819)
CVSSWGPESPYYFDYW;
(SEQ ID NO: 1820)
CAREYCTGGDCHFFLDYW;
or
(SEQ ID NO: 101)
QVWDSSSEHVV.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13102,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13108,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1817,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13120,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13126, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13103,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13109,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1818,
CDRL1 comprises an amino acid sequence at least 60% (for example, at least 60%, at least 65%, at least 70%, at least 75%, at least 80%, at least 85%, or at least 90%, at least 95%, at least 96%, at least 97%, at least 98%, or at least 99%) identical to SEQ ID NO: 13121,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13127, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13104,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13110,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1819,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13122,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13128, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13105,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13111,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1820,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13123,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13129, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13106,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13112,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 104 or 1821,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13124,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13130, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13107,
CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13113,
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 110 or 1822,
CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13125,
CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 13131, and/or
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 101 or 1888.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13102,
CDRH2 is SEQ ID NO: 13108,
CDRH3 is SEQ ID NO: 1817,
CDRL1 is SEQ ID NO: 13120,
CDRL2 is SEQ ID NO: 13126, and
CDRL3 is SEQ ID NO: 13 or 1883.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, CDRH3, wherein:
CDRH1 is SEQ ID NO: 13103,
CDRH2 is SEQ ID NO: 13109,
CDRH3 is SEQ ID NO: 1818,
CDRL1 is SEQ ID NO: 13121,
CDRL2 is SEQ ID NO: 13127, and
CDRL3 is SEQ ID NO: 25 or 1884.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13104,
CDRH2 is SEQ ID NO: 13110,
CDRH3 is SEQ ID NO: 1819,
CDRL1 is SEQ ID NO: 13122,
CDRL2 is SEQ ID NO: 13128, and
CDRL3 is SEQ ID NO: 39 or 1885.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13105,
CDRH2 is SEQ ID NO: 13111,
CDRH3 is SEQ ID NO: 1820,
CDRL1 is SEQ ID NO: 13123,
CDRL2 is SEQ ID NO: 13129, and
CDRL3 is SEQ ID NO: 49 or 1886.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13106,
CDRH2 is SEQ ID NO: 13112,
CDRH3 is SEQ ID NO: 104 or 1821,
CDRL1 is SEQ ID NO: 13124,
CDRL2 is SEQ ID NO: 13130, and
CDRL3 is SEQ ID NO: 88 or 1887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 13107,
CDRH2 is SEQ ID NO: 13113,
CDRH3 is SEQ ID NO: 110 or 1822,
CDRL1 is SEQ ID NO: 13125,
CDRL2 is SEQ ID NO: 13131, and
CDRL3 is SEQ ID NO: 101 or 1888.

In some embodiments, the method comprises administering to the subject the antibody disclosed in International Application Publication No. WO2020/206232, which is incorporated herein by reference in its entirety.

In some embodiments, the antibody described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

As the timing of an infection or onset of a related disease can often not be predicted, it should be understood the disclosed methods of treating, inhibiting, reducing, ameliorating, and/or preventing the disease or disorder described herein can be used prior to or following the onset of the infection or the disease, to treat, prevent, inhibit, and/or reduce the infection or the disease. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the infection or the disease; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the infection or the disease.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Introduction

Human immunodeficiency virus (HIV-1) and hepatitis C virus (HCV) are two of the most diverse human pathogens, ever-evolving to evade immune system pressure, typically establishing chronic, life-long infection. Furthermore, due to the shared routes of transmission, HIV-1/HCV co-infection is relatively common, affecting an estimated 5 million individuals worldwide. Although the last 30+ years have seen significant advances in the treatment of both viruses, there are still no licensed vaccines or other prophylactic countermeasures. Moreover, there is a cure for HCV available, yet less than 50% of infected individuals know of their positive status, highly limiting its utility. Poor medication and diagnostic access, as well as high re-infection rates, for which HIV-1/HCV co-infected individuals experience the highest re-infection rates with either virus, strongly motivate the development of alternative therapeutic and prophylactic tools. Such new tools will be of important utility in the setting of HIV-1/HCV co-infection, where the chronic exposure to two mutating pathogens leads to significantly exacerbated health problems compared to mono-infection.

Although these highly mutable viruses have rendered classical vaccine design difficult, investigating the human antibody response to HIV-1 and HCV mono-infection has led to the identification of antibodies that are effective in therapy and prophylaxis, and that have served as templates for antibody-specific vaccine development. The clinical setting of HIV-1/HCV co-n infection has been far less explored, with little understanding about antibody responses in the chronic presence of two diverse, constantly evolving, antigen targets. This study investigates the antibody repertoire of a chronically HIV-1/HCV co-infected individual using LIBRA-seq, a technology that enables the simultaneous screening of B cells against a diverse library of antigen targets. This study shows that LIBRA-seq identified antibodies with binding and functional cross-reactivity between HIV-1 and HCV, without exhibiting typical traits of promiscuous antigen recognition. These results challenge the long-standing understanding of the exclusiveness of antibody-antigen specificity and pave the way toward the development of effective therapeutics and vaccines with an unparalleled breadth of reactivity.

### Example 2. Results

Discovery of HIV-1/HCV Cross-reactive Antibodies from a Chronically HIV-1/HCV Co-infected Donor. To probe the development of antibody responses produced by the unique immunological challenge of HIV-1/HCV co-infection, the antigen-specific B cell compartment was profiled using LIBRA-seq. As previously described, LIBRA-seq is a technology that allows for high-throughput mapping of antigen specificity to B cell receptor sequence by leveraging oligo-barcoded antigens and single-cell sequencing. A donor, VC10014 was identified from the Vanderbilt HIV-1 infection cohort, who had been chronically HIV-1/HCV co-infected for >3 years at the time of sample collection and had never taken anti-viral or anti-retroviral medication. Previous studies investigating key events leading to early development of broad HIV-1 neutralization established that VC10014 developed broad serum neutralization approximately one year after HIV-1 infection and that this phenotype can largely be traced to a CD4 binding site-directed antibody response. Monoclonal antibody discovery efforts in this donor failed to identify any broadly neutralizing antibodies, instead attributing the observed serum breadth to a diverse but cooperative antibody lineages. Given the ability of LIBRA-seq to screen tens of thousands of B cells against a large panel of diverse antigens, including those from unrelated pathogens, it was sought to apply this technology to HIV-1/HCV co-infected donor VC10014. The application of LIBRA-seq provides a unique opportunity to interrogate the antibody repertoire in the setting of chronic exposure to diverse—and constantly evolving—antigens.

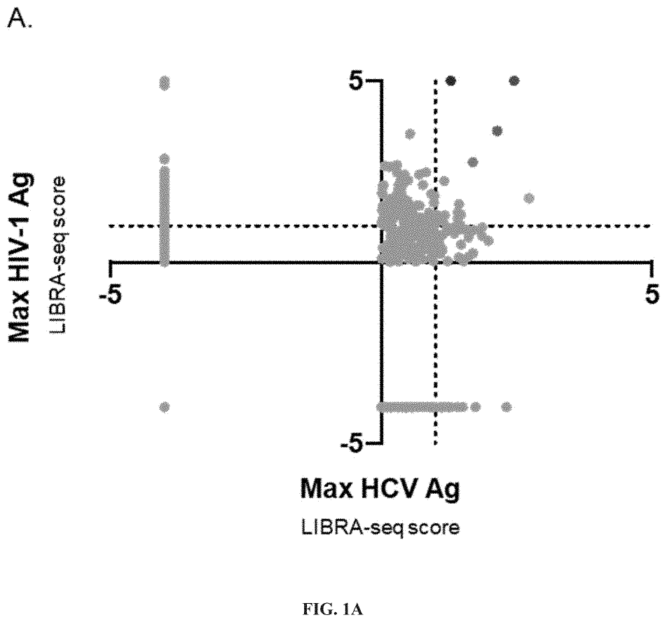

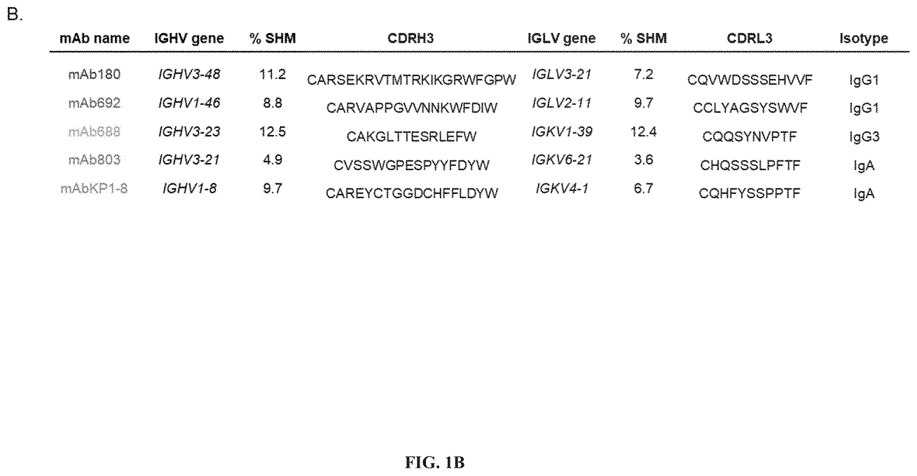

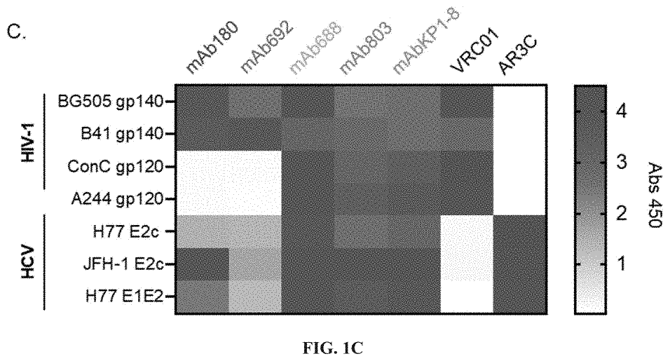

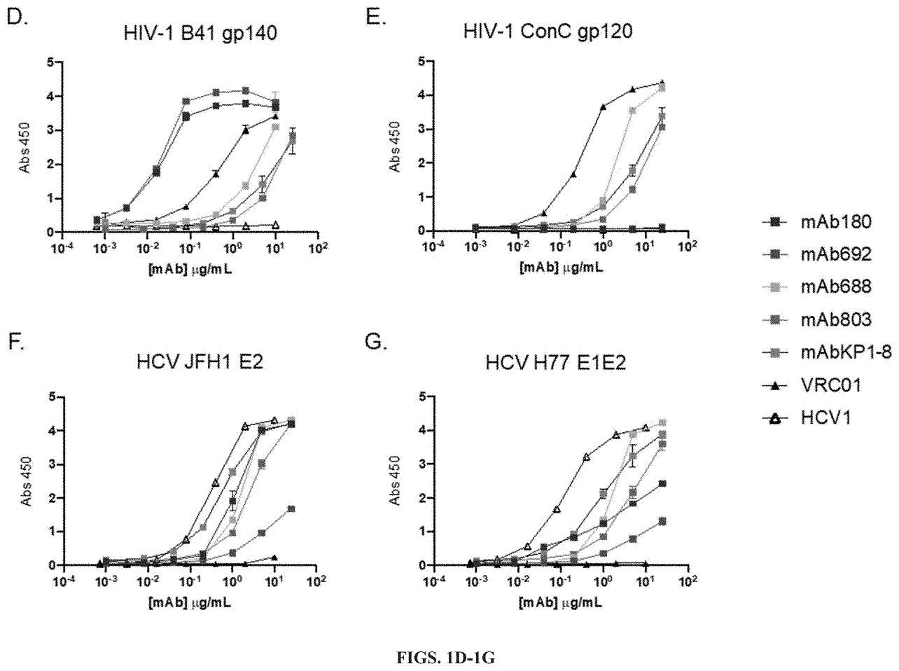

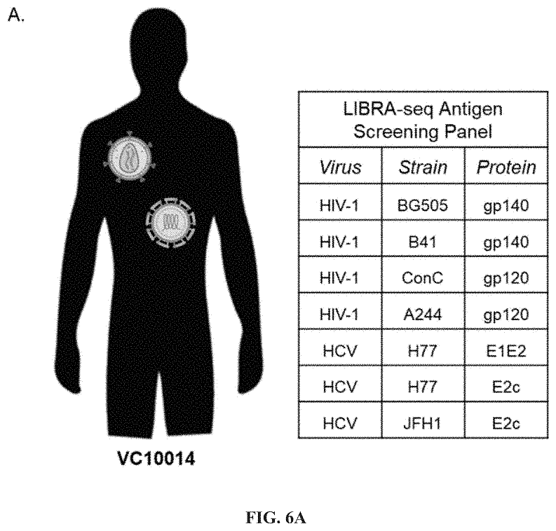

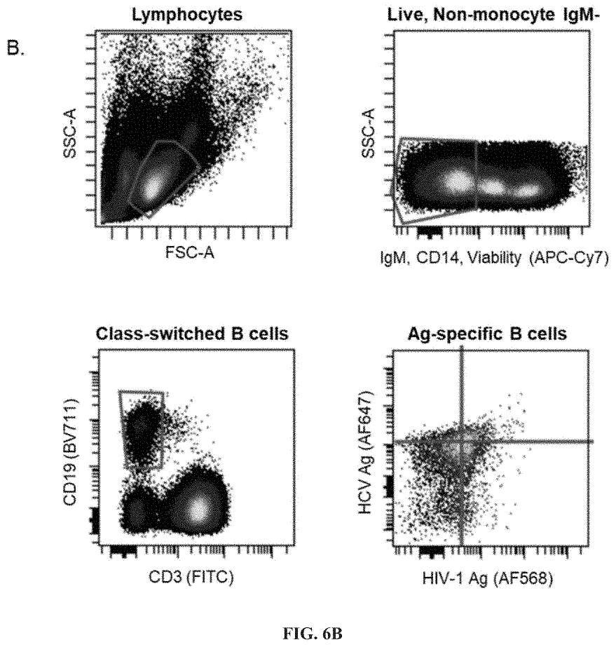

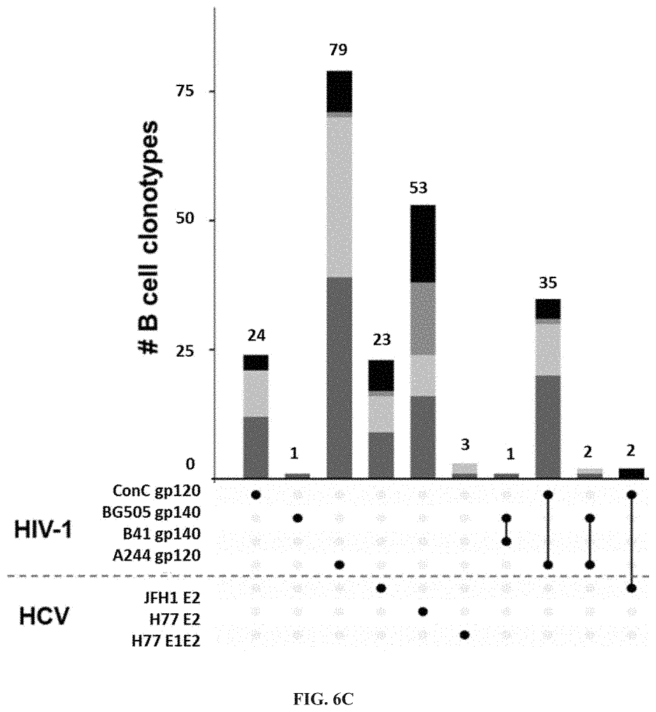

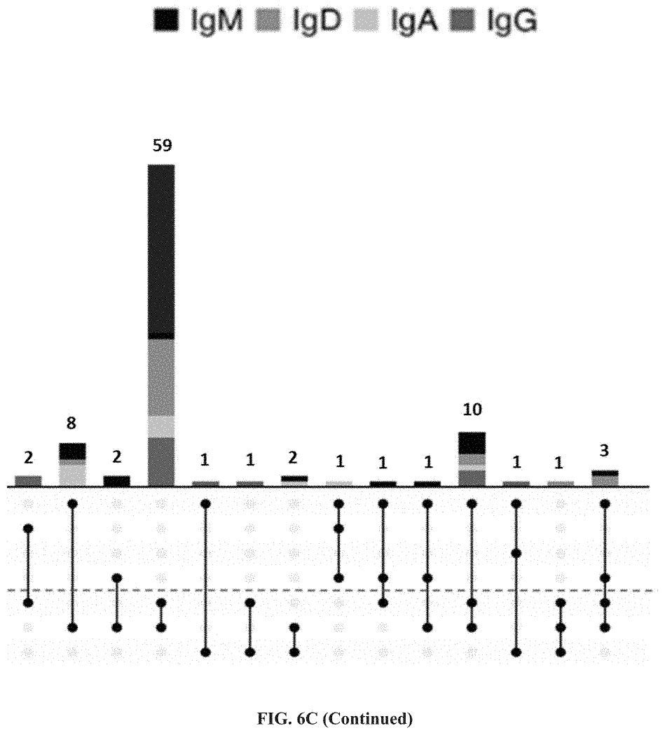

To identify virus-specific B cell sequences from VC10014, LIBRA-seq was applied with a diverse panel of seven antigens including four HIV-1 envelope (Env) glycoprotein antigens each from a unique clade (A/BG505 gp140, B/B41 gp140, C/ConC gp120, AE/A244 gp120), and three HCV envelope glycoprotein antigens from two distinct genotypes (1a/H77 E2c, H77 E1E2, and 2a/JFH1 E2c) (FIG. 6A). The inclusion of multiple antigen variants for each of HIV-1 and HCV allows for the identification of antibodies with broad diversity of antigen specificities. LIBRA-seq recovered paired VH:VL B cell receptor (BCR) sequences with associated antigen specificity mapping for 886 cells (FIGS. 6B, 6C). Of the identified class-switched BCR sequences, ˜75% were HIV-1 antigen-specific, and ˜23% were HCV antigen-specific. Interestingly, a small population of cells positive for at least one HIV-1 and at least one HCV antigen were also identified (FIG. 6C). Within this population of cross-reactive B cells, the class-switched cells with the highest LIBRA-seq scores for any HIV-1 and HCV antigen were assessed (FIG. 1A). The analysis identified five genetically unique sequences with varied levels of somatic hypermutation (SHM) and HIV-1 antigen+/HCV antigen+ specificity phenotypes for further study (FIG. 1B). These five paired heavy-light chain sequences were expressed as recombinant antibodies and their reactivity against a panel of HIV-1 and HCV envelope glycoproteins was confirmed by ELISA (FIGS. 1C-1G). The results confirmed that LIBRA-seq successfully indicated the HIV-1/HCV envelope antigen cross-reactivity of the identified antibodies.

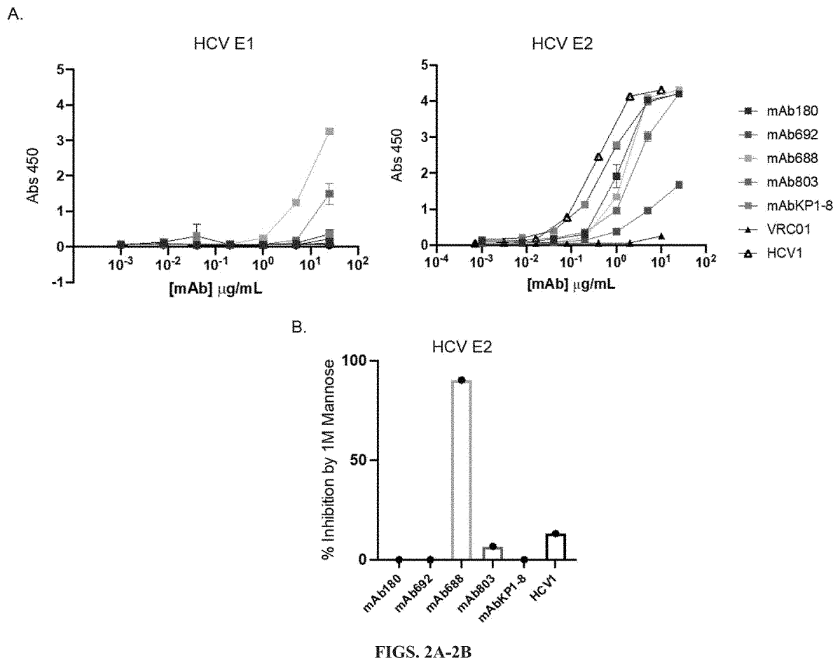

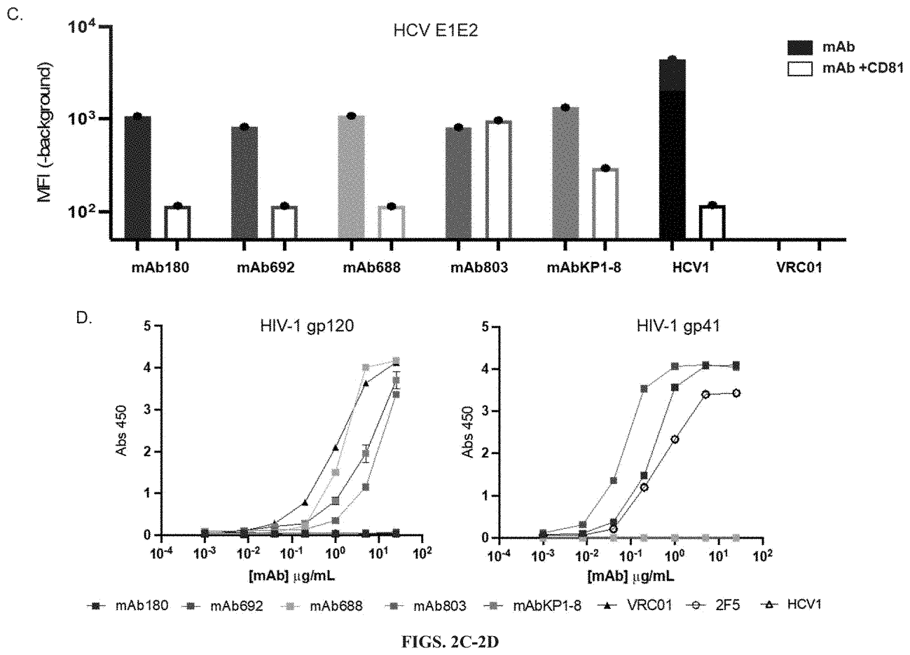

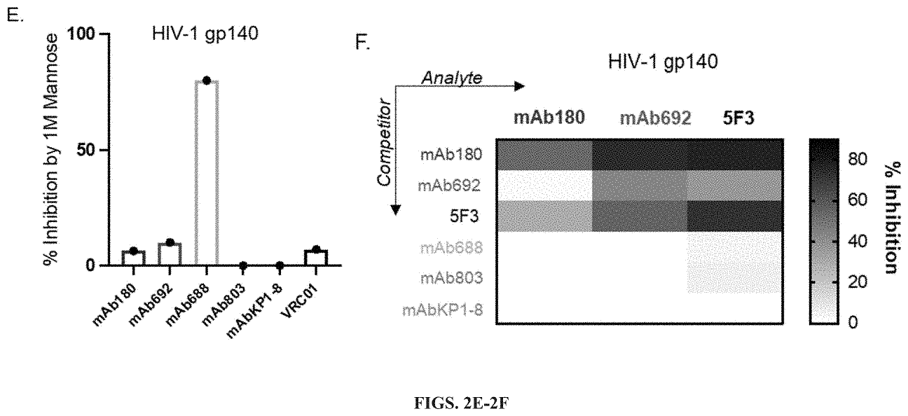

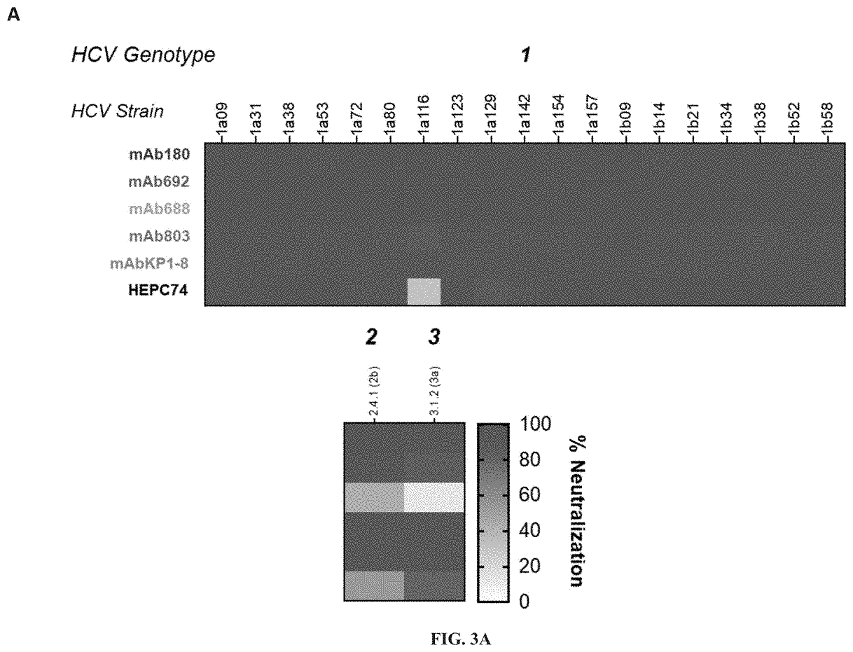

HIV-1/HCV Cross-reactive Antibodies Recognize Distinct Epitopes on the HIV-1 and HCV Envelope Glycoproteins. Given the unique antibody-antigen cross-reactivity, the epitope of these antibodies on the two antigen targets was mapped. The five cross-reactive antibody epitope targeting on the HCV envelope was defined. All antibodies bound recombinant HCV E1E2 with recognition directed to the E2 subunit of the glycoprotein, with only mAb688 displaying appreciable reactivity with E1 (FIGS. 1F-1G, FIG. 2A). To further map the epitopes targeted, this experiment measured antibody competition with CD81, the cognate HCV entry receptor (FIG. 2C). Binding of four of the five HIV-1/HCV cross-reactive antibodies was inhibited by pre-incubation with CD81-LEL (FIG. 2C). mAb803 binding was slightly increased in the presence of receptor (FIG. 2C).

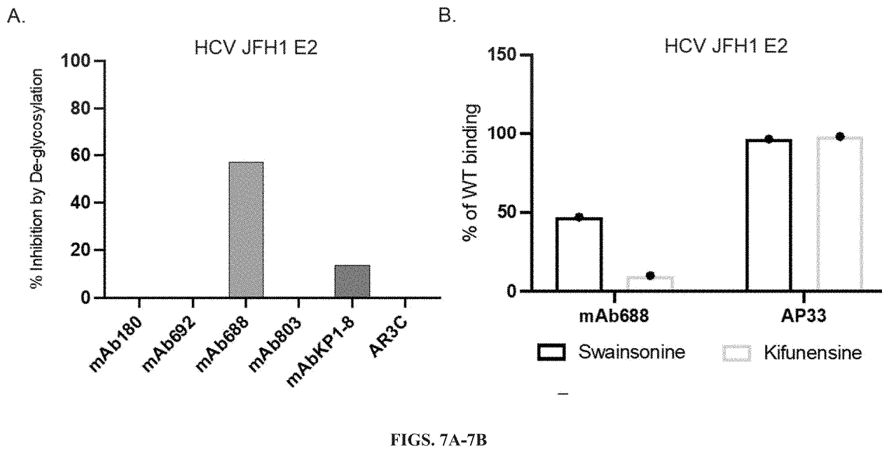

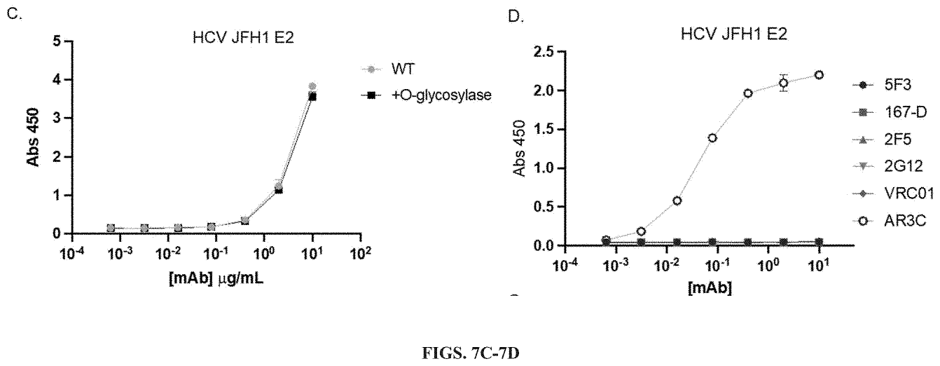

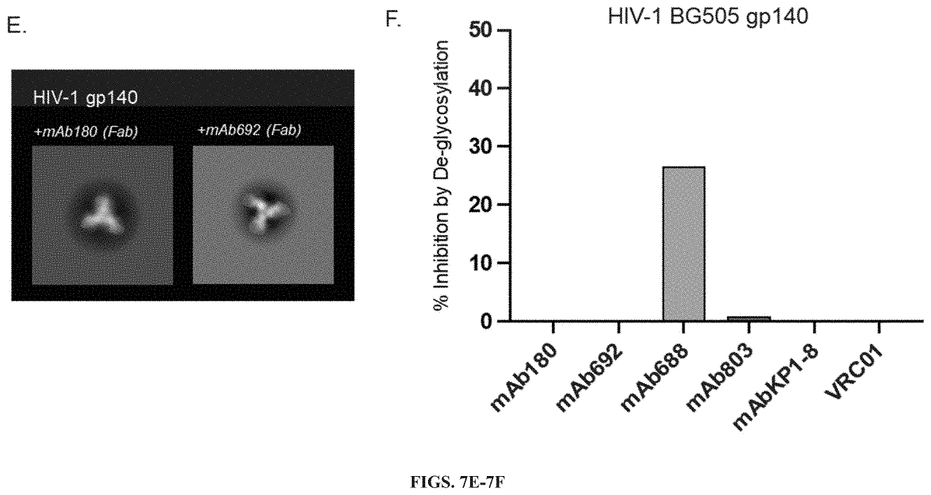

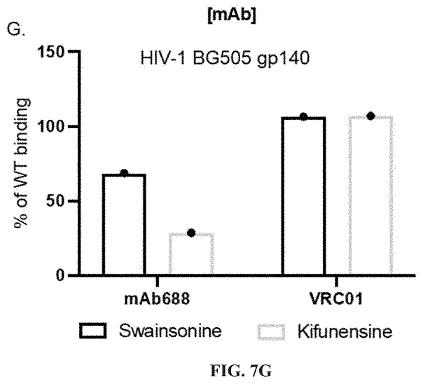

Given the extensive glycan shield that decorates both the HIV-1 and HCV envelope glycoproteins, the glycan-dependence of HIV-1/HCV cross-reactive recognition was defined. Only mAb688 was inhibited by both 1M D-(+)-mannose and PNGaseF de-glycosylation (FIG. 2B, FIG. 7A). Additionally, in experiments with HCV E2 protein produced in the presence of kifunensine, a class I α-mannosidase inhibitor that results in the majority presence of high mannose-type glycans, mAb688 binding was decreased, indicating that recognition requires additional contacts or glycan processing (FIG. 7B). In summary, HIV-1/HCV cross-reactive antibodies recognize at least three distinct epitopes on the HCV envelope protein including CD81-independent (mAb803), as well as glycan-dependent (mAb688) and -independent (mAb180, mAb692, mAbKP1-8) CD81-blocking regions.

Next, the epitopes of these antibodies on the HIV-1 envelope protein were mapped. All five antibodies bound soluble HIV-1 gp140, albeit to various degrees (FIG. 1D). Two of the five antibodies (mAb180, mAb692) recognized the gp41 subunit of HIV-1 Env, while the other three antibodies recognized gp120 (FIG. 2D, FIG. 7C). In competition ELISA experiments it was found that mAb180 and mAb692 recognized epitopes overlapping with that of 5F3 (FIG. 2F). 5F3 has previously been reported to interact with both the C-terminal heptad repeat region (CHR) and the fusion peptide proximal region (FPPR). Importantly, binding of 5F3 or related CHR/FPPR-specific HIV-1 antibodies (167-D, F240) to the HCV envelope protein was not detected, indicating these LIBRA-seq-identified antibodies represent a unique binding modality (FIG. 7D). mAbs 688, 803, and KP1-8 did not compete with the gp41-reactive mAb180 and mAb692 for binding to HIV-1 gp140 (FIG. 2F).

As with HCV, glycan-dependence of antibody binding to HIV-1 gp140 was investigated. HIV-1 gp140 is a viral protein ornamented with under-processed glycans. Only mAb688 binding to HIV-1 gp140 was inhibited by PNGaseF de-glycosylation and competition with 1M D-(+)-mannose (FIG. 2E, FIG. 7F). Similar to experiments with HCV E1E2, mAb688 recognition of HIV-1 gp140 was significantly decreased after treatment with the class I α-mannosidase inhibitor kifunensine (FIG. 7G). Taken together, these data support the discovery of antibodies recognizing at least three distinct regions on the HIV-1 Env glycoprotein encompassing gp41 (mAb180, 692), and both glycan-dependent (mAb688) and -independent (mAb803, KP1-8) gp120 epitopes.

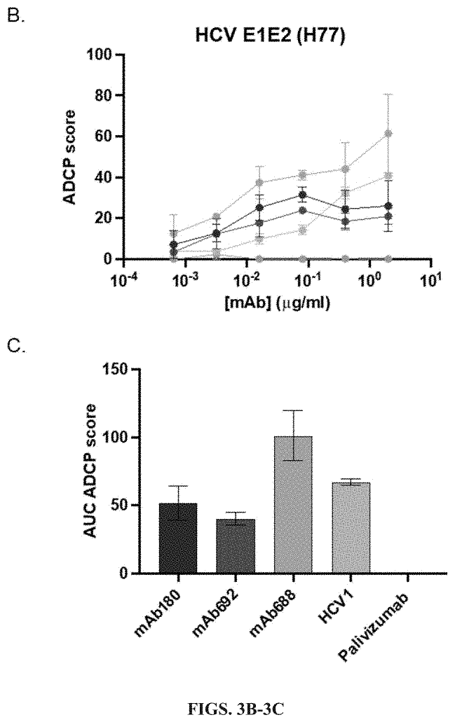

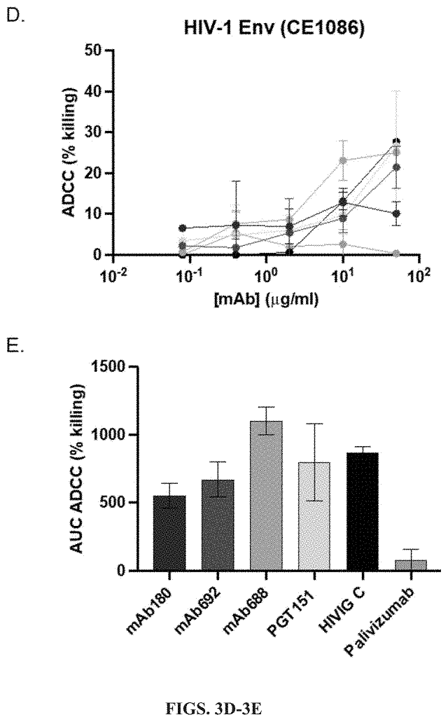

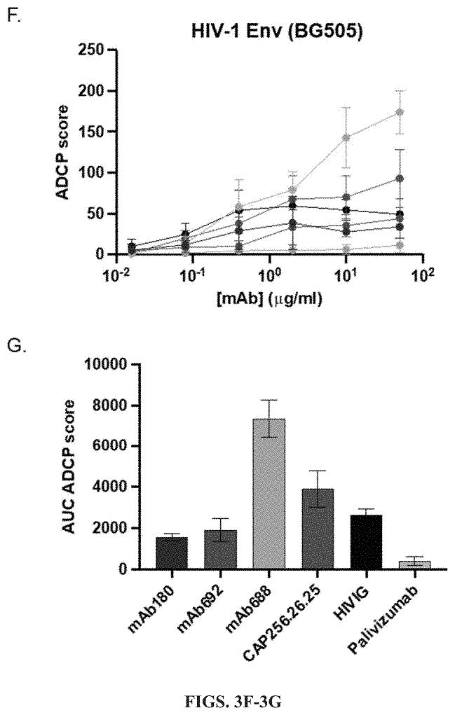

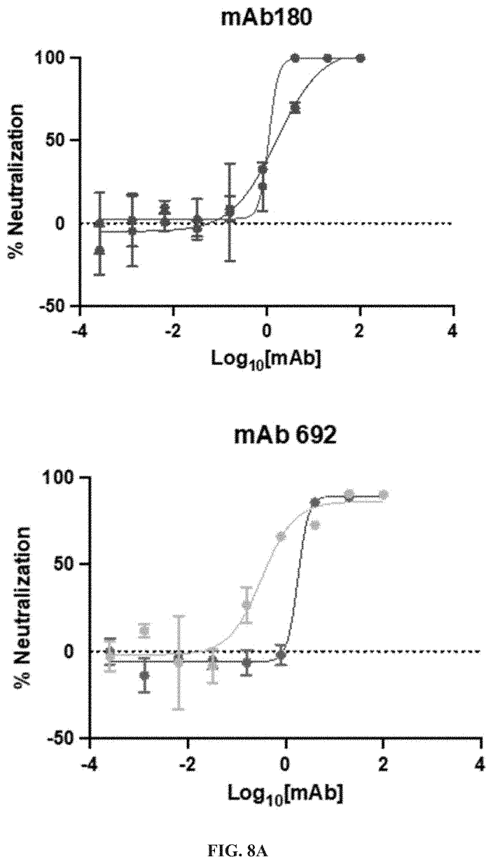

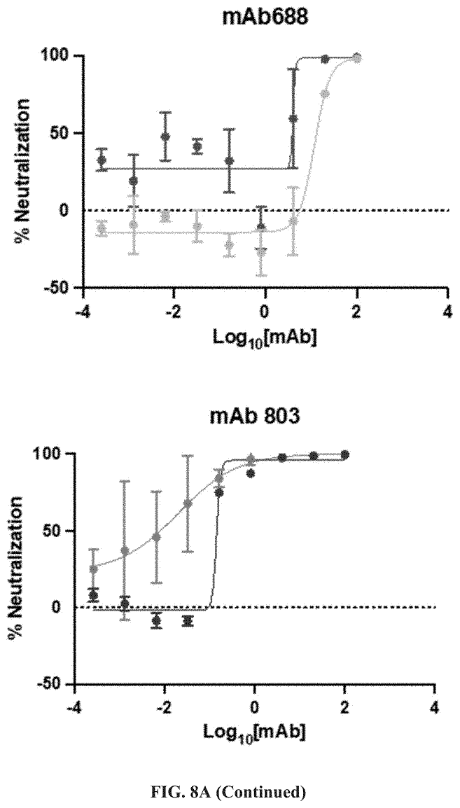

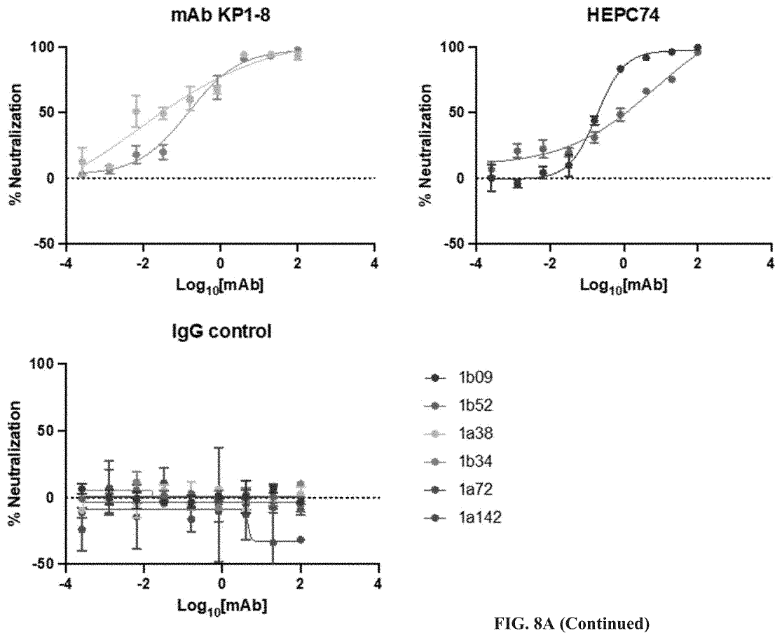

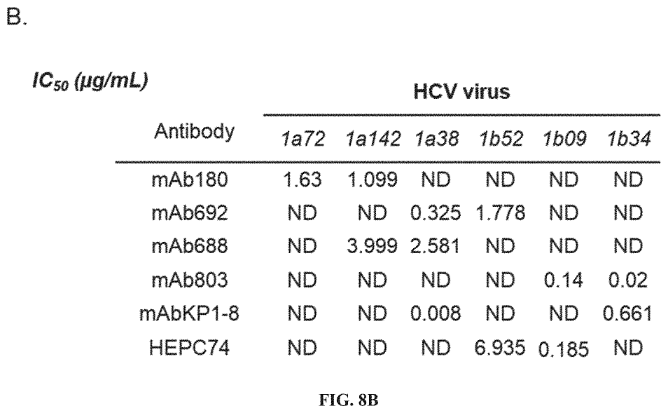

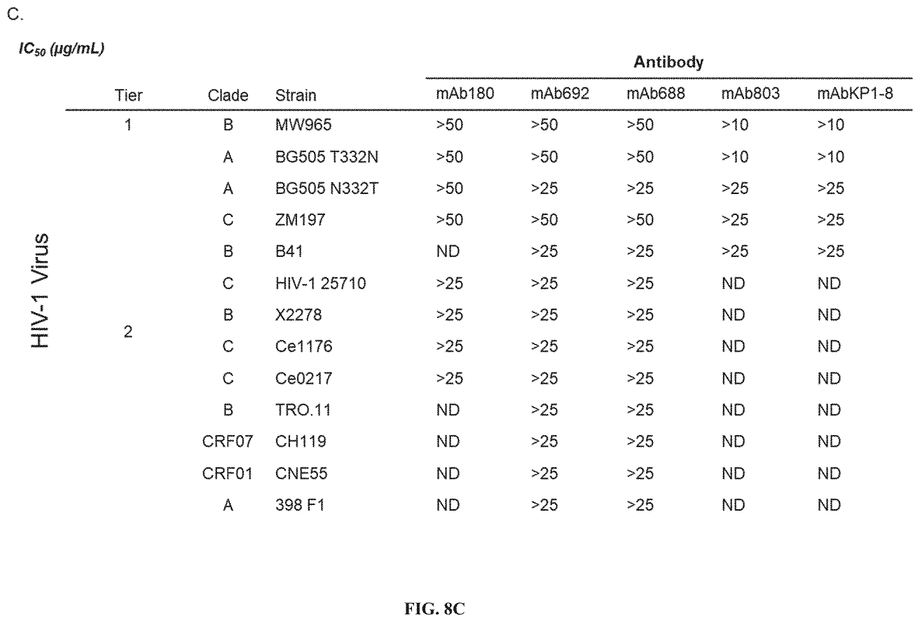

HIV-1/HCV Cross-reactive mAbs Show Diverse Neutralization and Fc-mediated Effector Functions. After evaluating HIV-1 and HCV antigen specificity, next experiment assessed the functional abilities of the HIV-1/HCV cross-reactive antibodies in both neutralization and Fc-mediated effector function assays. First, the ability of HIV-1/HCV cross-reactive antibodies to neutralize a panel of representative genotype 1 HCV strains was investigated. Notably, all five HIV-1/HCV cross-reactive antibodies showed exceptional HCV neutralization breadth, neutralizing all 19 genotype 1 viruses tested at 100 μg/mL (FIG. 3A, FIGS. 8A and 8B). This is particularly striking when compared to the previously reported broadly neutralizing antibodies HEPC74 or AR3C, which neutralized 18/19 or 16/19 genotype 1 viruses in this panel, respectively. Further, four of the five HIV-1/HCV cross-reactive antibodies were also able to neutralize both genotype 2b and 3a strains in an infectious cell culture-generated virus (HCV_{CC}) assay (FIG. 3A). Anti-HCV function for these antibodies was measured. Whether the IgG antibodies mAb180, mAb692, and mAb688 can mediate anti-HCV E1E2 Fc effector functions was tested. All tested antibodies (mAb180, mAb692, mAb688) mediated antibody-dependent cellular phagocytosis (ADCP) against HCV E1E2 (genotype 1a; strain H77) (FIGS. 3B, 3C).

Next, the anti-HIV-1 functions of HIV-1/HCV cross-reactive antibodies was characterized and none of the antibodies showed neutralizing activity against the HIV-1 strains tested (FIG. 8C). However, all tested antibodies (mAb180, mAb692, mAb688) were capable of potentiating antibody-dependent cellular cytotoxicity (ADCC) against infectious HIV-1 envelope (strain CE1086) (FIGS. 3D, 3E). Moreover, all three antibodies mediated antibody-dependent cellular phagocytosis (ADCP) against HIV-1 gp140 (strain BG505) (FIGS. 3F, 3G). Taken together, beyond binding of diverse viral envelope glycoproteins, the identified antibodies revealed extraordinary cross-functionality. The IgA isotype antibodies (mAb803, KP1-8) were not tested in the Fc effector assays against either virus, as this isotype plays a less significant role in serological anti-viral Fc-mediated immunity.

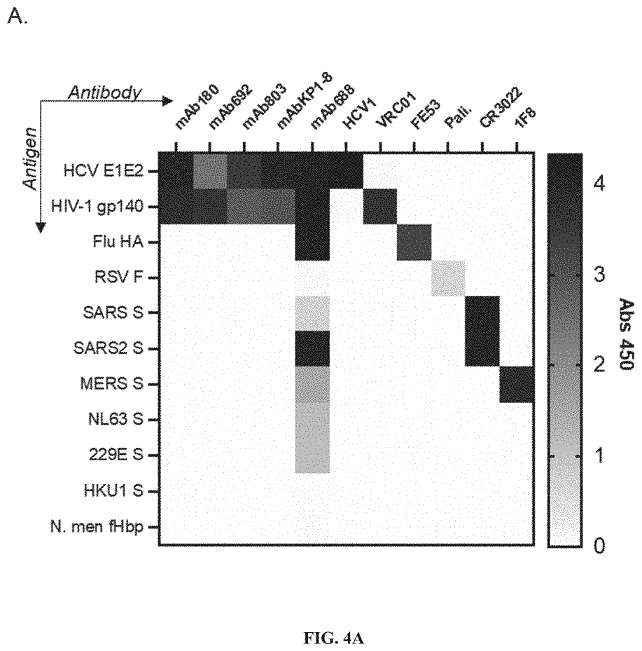

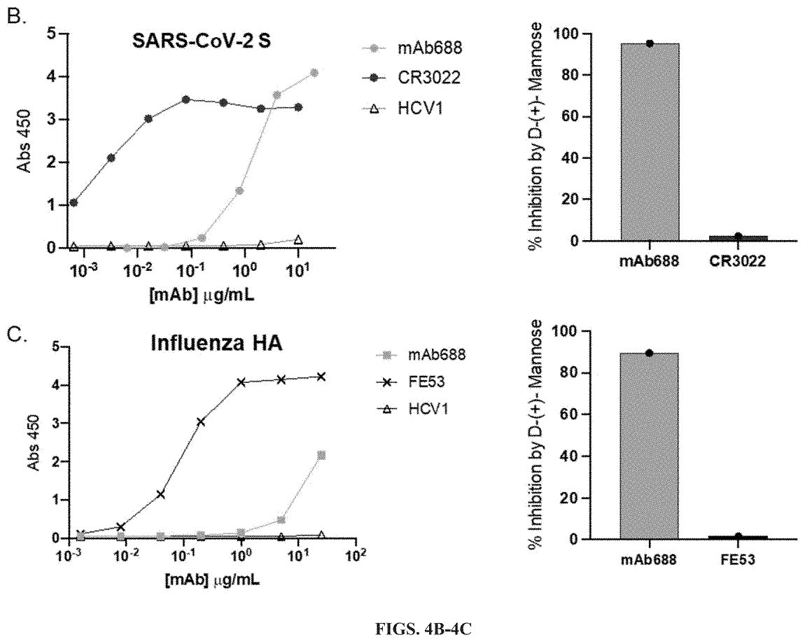

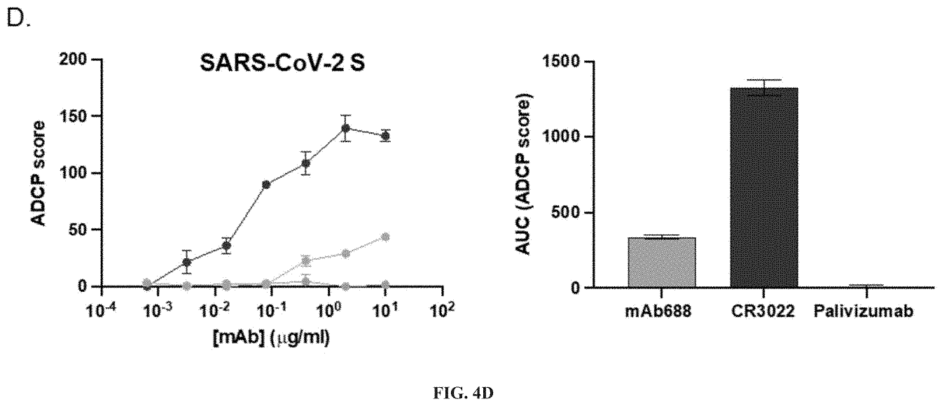

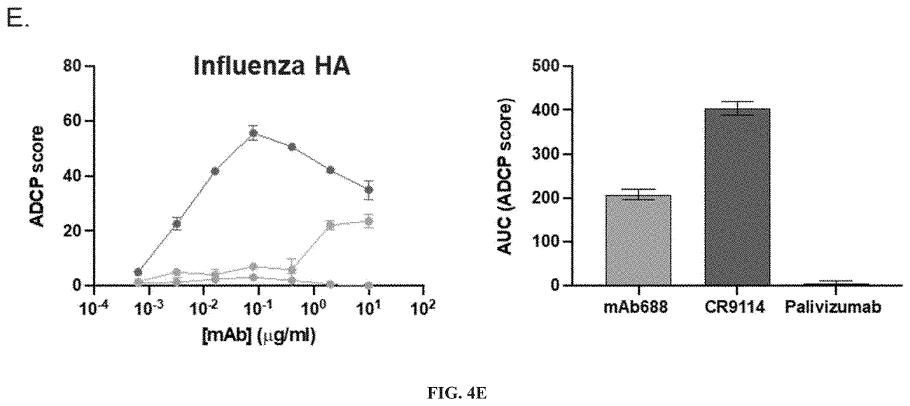

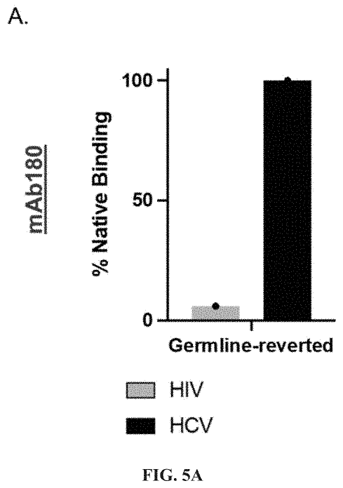

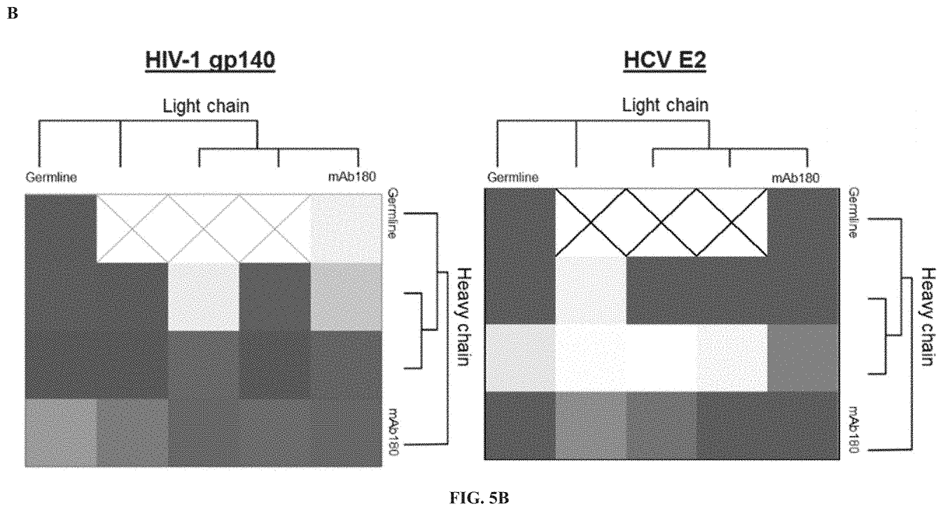

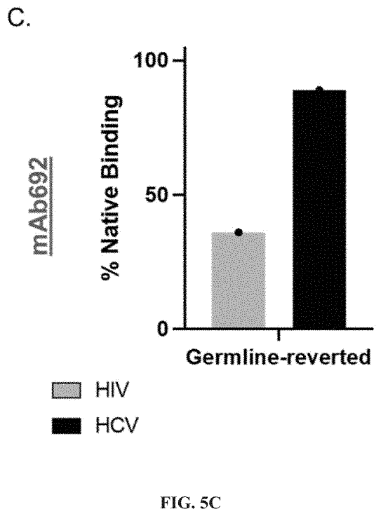

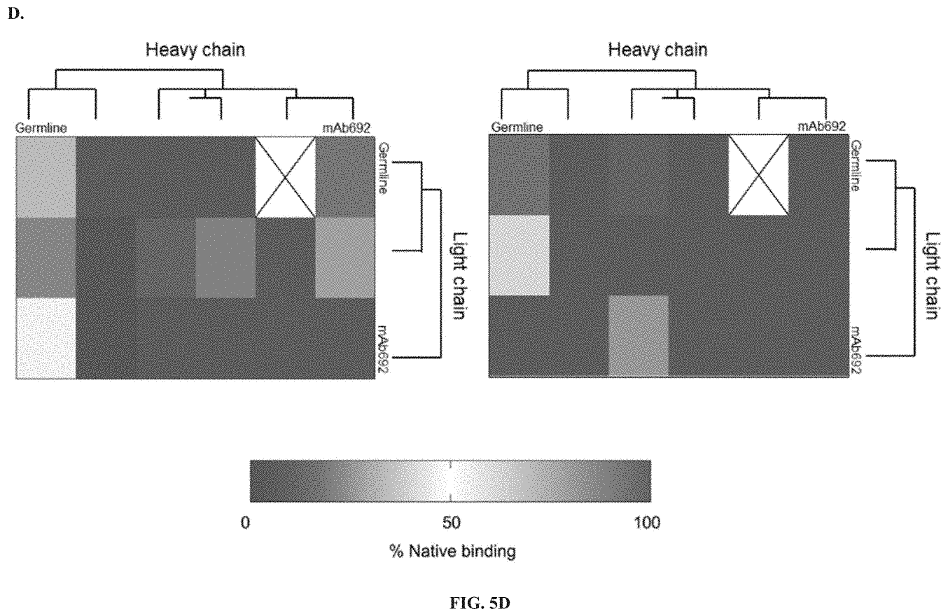

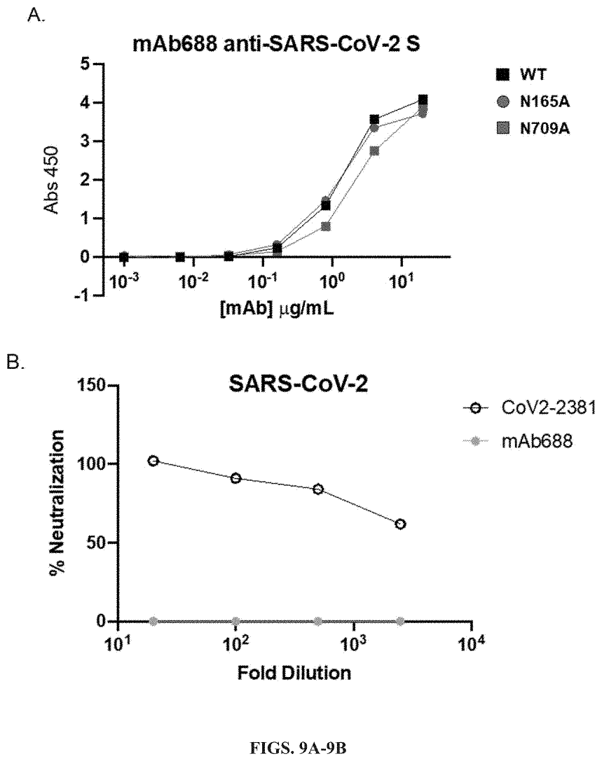

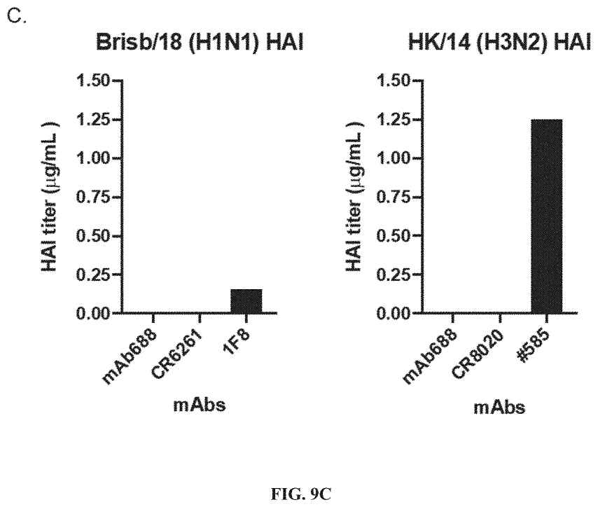

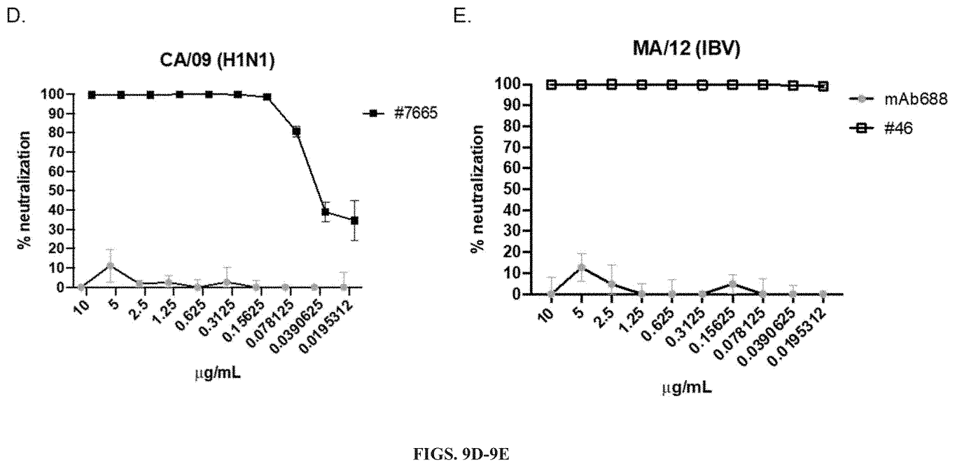

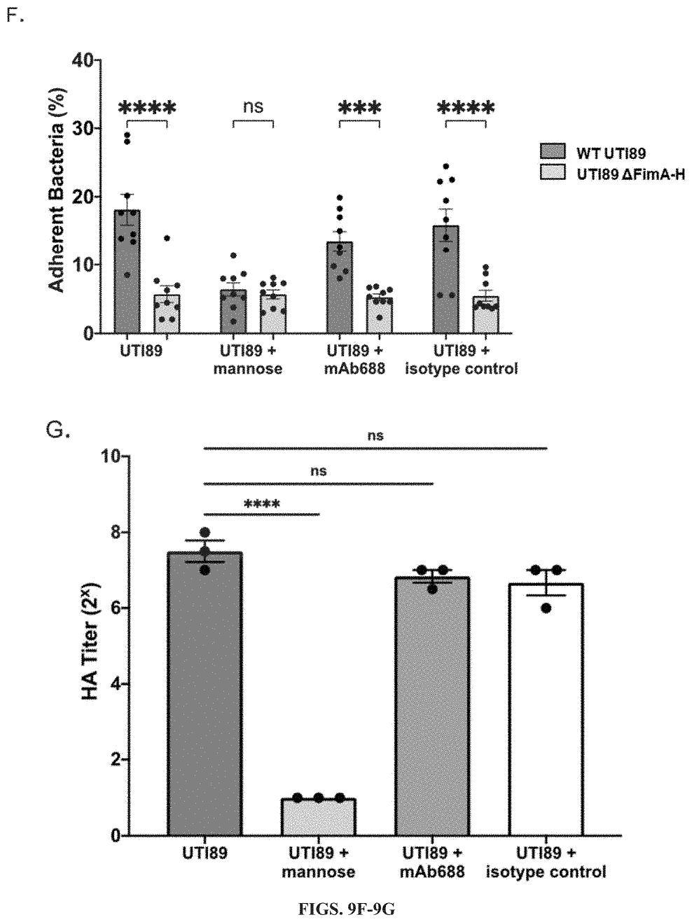

mAb688 Reveals Exceptionally Broad Anti-Viral Functions. Next assessed whether the LIBRA-seq-identified antibodies were solely HIV-1/HCV cross-reactive, or whether they can recognize additional viral envelope glycoproteins. To that end, these antibodies were tested against a panel of antigens from a diverse set of pathogens, and found that mAb 180, 692, 803, and KP1-8 indeed bound only the HIV-1 and HCV antigens tested (FIG. 4A). By contrast, mAb688 recognized a broad diversity of other viral antigens including glycoproteins from Influenza A, alphacoronaviruses NL63 and 229E, and betacoronaviruses MERS-CoV, SARS-CoV, and notably, SARS-CoV-2 (FIG. 4A). As observed with HIV-1 and HCV, it was found that mAb688 recognition of both influenza A hemagglutinin and SARS-CoV-2 spike was mannose-dependent (FIGS. 4B, 4C). Previous studies indicated that the anti-HIV-1 antibody 2G12 is also able to bind the SARS-CoV-2 spike, by recognition of a high mannose glycan structure on asparagine 709. Comparatively, mAb688 binding to SARS-CoV-2 spike was not inhibited by N709A mutation (FIG. 9A). mAb688 was unable to neutralize SARS-CoV-2 but showed ADCP activity against the SARS-CoV-2 spike protein (FIG. 4D, FIG. 9B). Similarly, mAb688 was unable to neutralize influenza virus but showed ADCP against the influenza A hemagglutinin protein (FIG. 4E, FIGS. 9C-9E). Together with the mAb688 HIV-1 and HCV functional activity, these data indicate that mAb688 is capable of diverse functions against a broad range of viral targets.

Finally, to determine whether the observed broad functional abilities of mAb688 spanned beyond virus targets, whether mAb688 can inhibit the most common etiological agent of urinary tract infection (UTI), Uropathogenic Escherichia coli (UPEC), was investigated. UPEC potentiates infection using fimbriae to recognize mannosylated bladder host cell surface glycoprotein and red blood cells. As this interaction between bacteria and host is free mannose-inhibitable, whether mAb688 recognition of host mannose can block function was tested. Importantly, neither mAb688 nor the isotype control were able to impede UPEC adherence or hemagglutination (FIGS. 9F, to 9G), indicating that mAb688 recognizes a mannose structure that can be specific to viral glycosylation and/or requires additional antigen interactions.

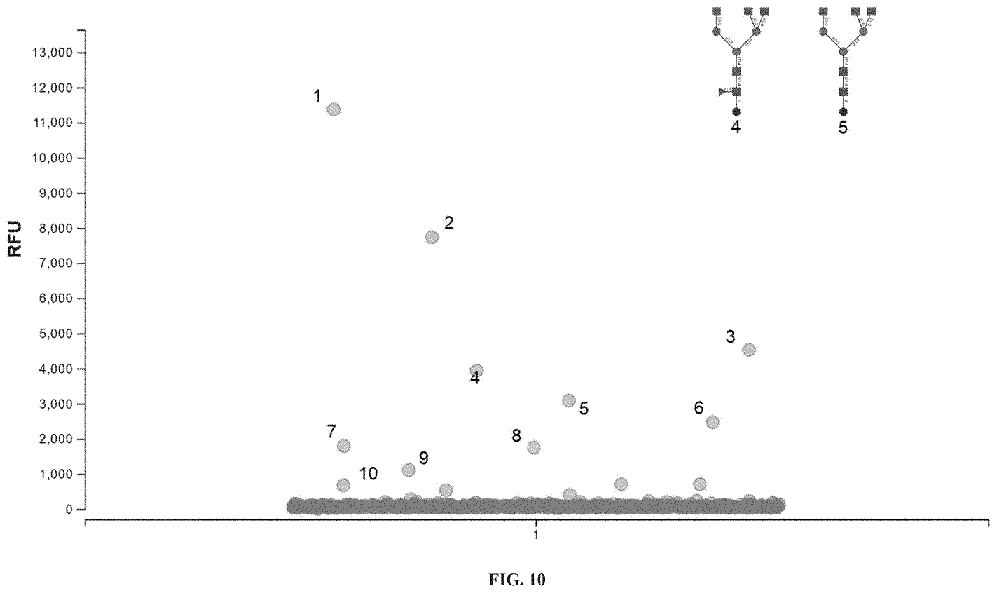

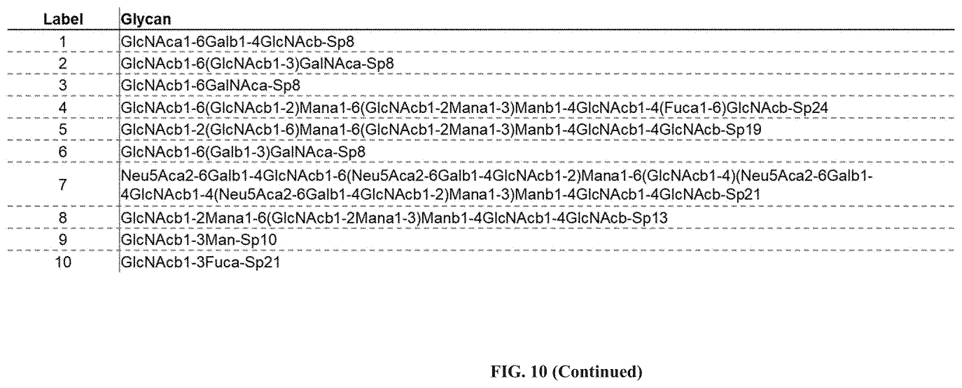

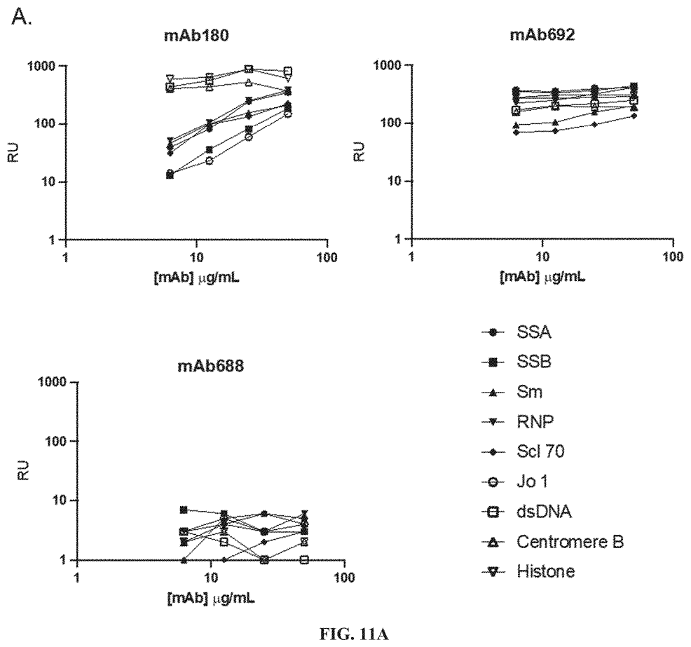

Finally, to define specific glycan architecture that mediates broad mAb688 recognition, this study tested binding to a glycan microarray consisting of >580 distinct structures developed by the Center for Functional Glycomics (CFG, v5.4 microarray). The majority of observed glycan hits contained a terminal N-acetyl glucosamine with (31-6 linkage with mannose or galactose, suggesting this is critical for mAb688 binding (FIG. 10). Further, these data demonstrate mAb688 preferentially recognizes immature, hybrid-type glycans, a form of glycosylation that's enriched on viral glycoproteins (FIG. 10).

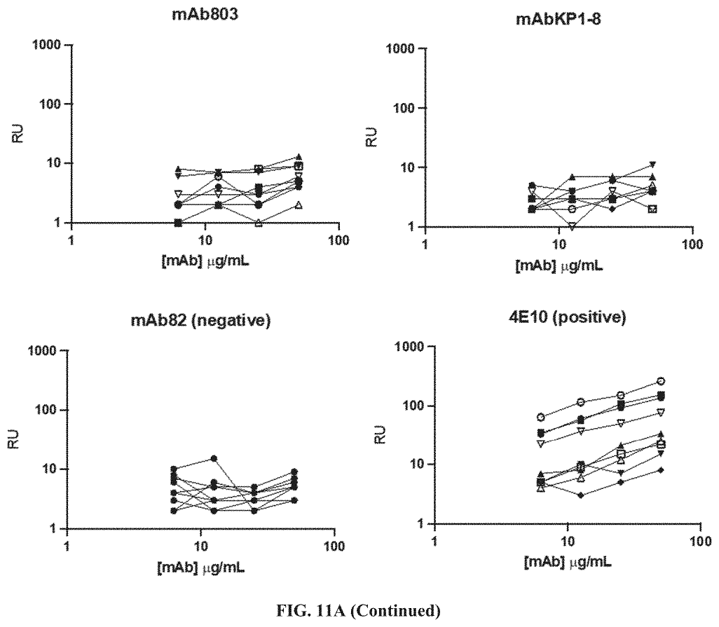

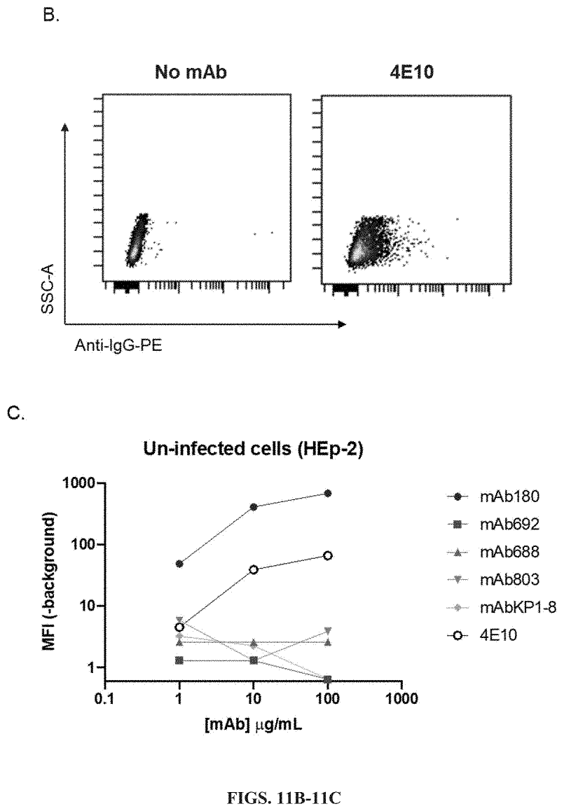

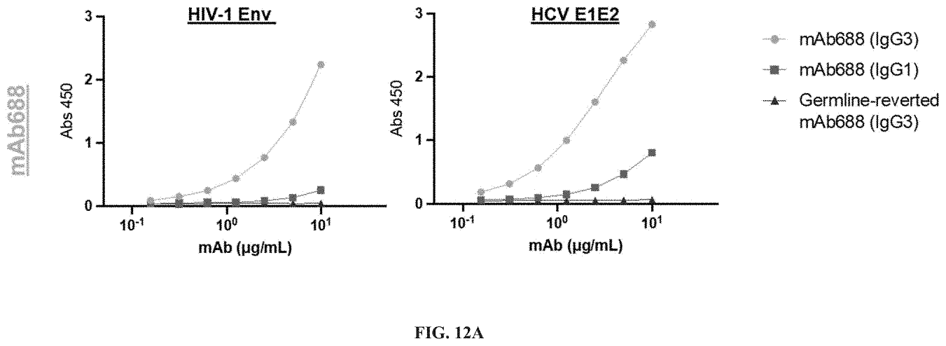

Diverse polyreactivity profiles of HIV-1/HCV cross-reactive mAbs. To investigate whether the cross-reactive antibodies can achieve diverse binding phenotypes via antigen polyreactivity, or non-specific interactions, reactivity to a panel of nuclear self-antigens was measured using the Luminex AtheNA Multi-analyte ANA assay (FIG. 11A). Similar to previously described gp41 antibodies, autoreactivity was observed for both mAb180 and mAb692. Further, mAb180, but not the other antibodies, bound non-infected, whole (un-permeabilized) HEp-2 cells in a fluorescent assay (FIGS. 11B, 11C). Therefore, from the set of five cross-reactive antibodies, only mAb180 and mAb692 showed binding in polyreactivity assays, indicating that the broadly reactive anti-viral phenotype of the other antibodies—including the exceptionally broad mAb688—cannot be explained by promiscuous, non-specific, antigen interactions.

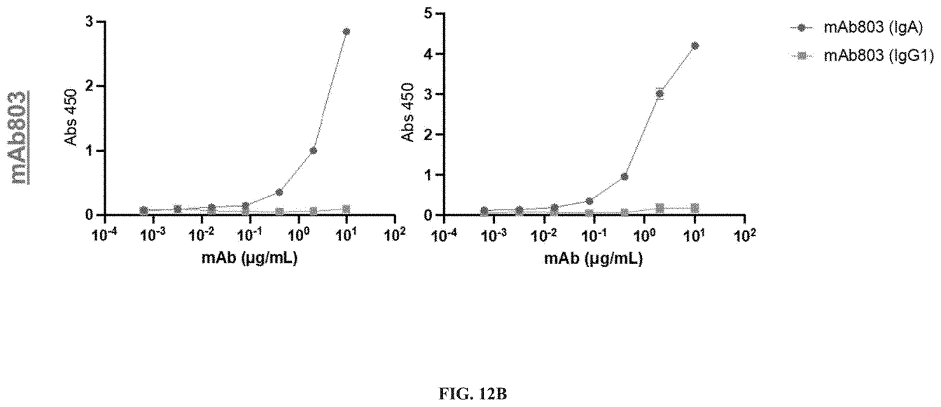

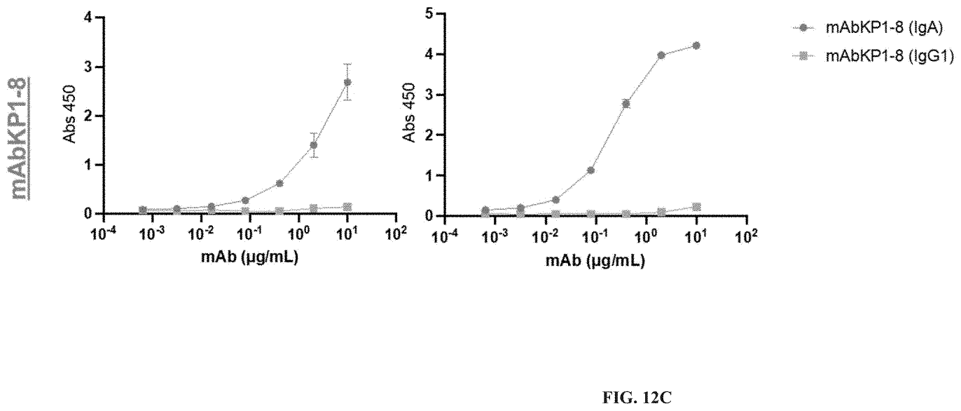

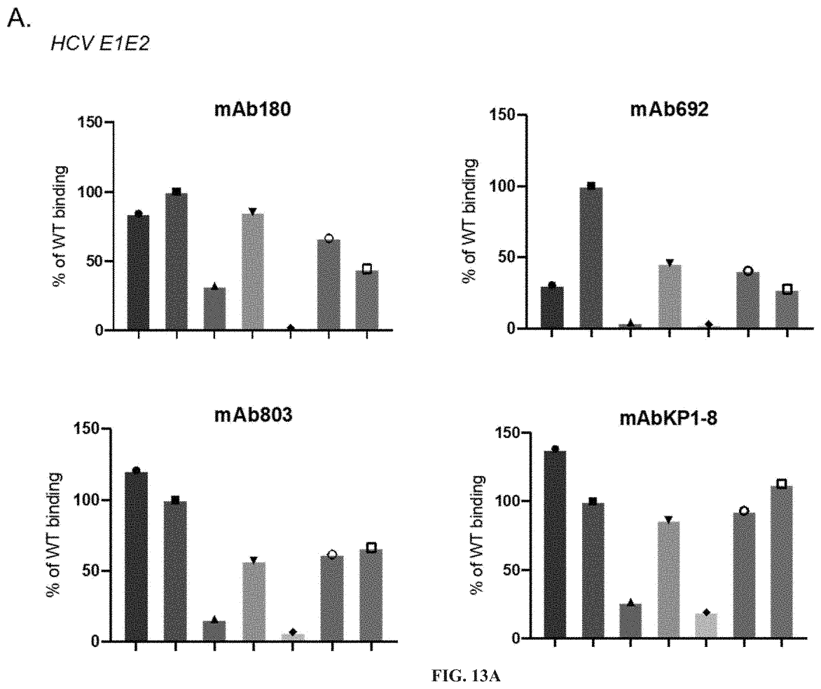

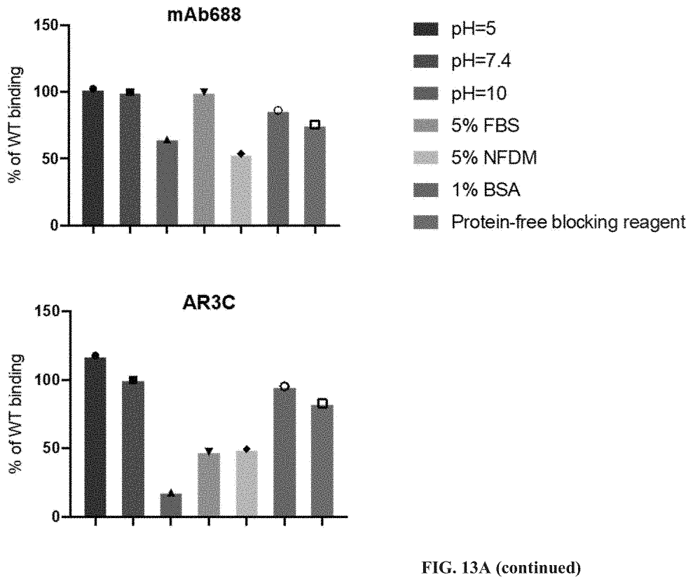

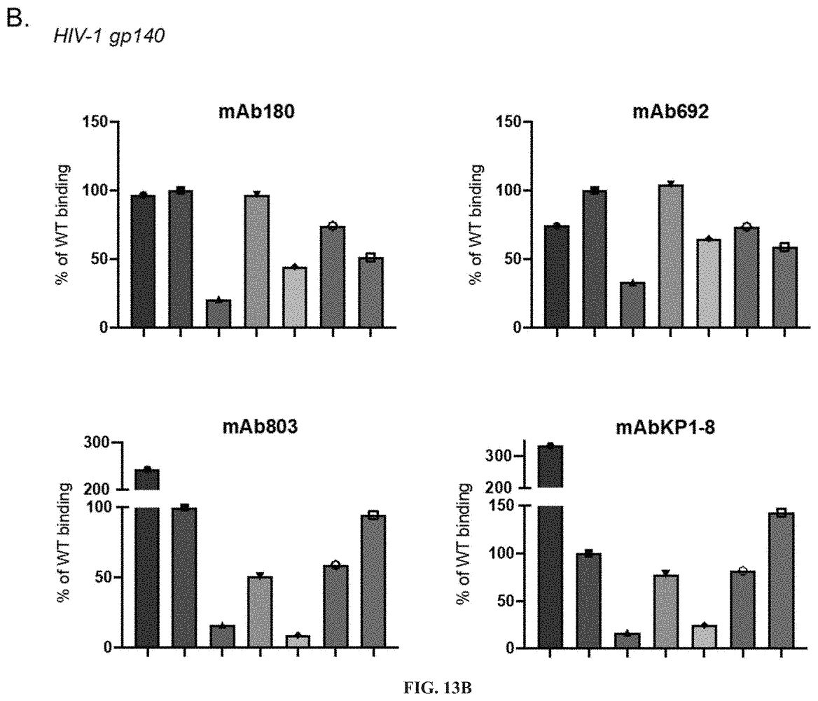

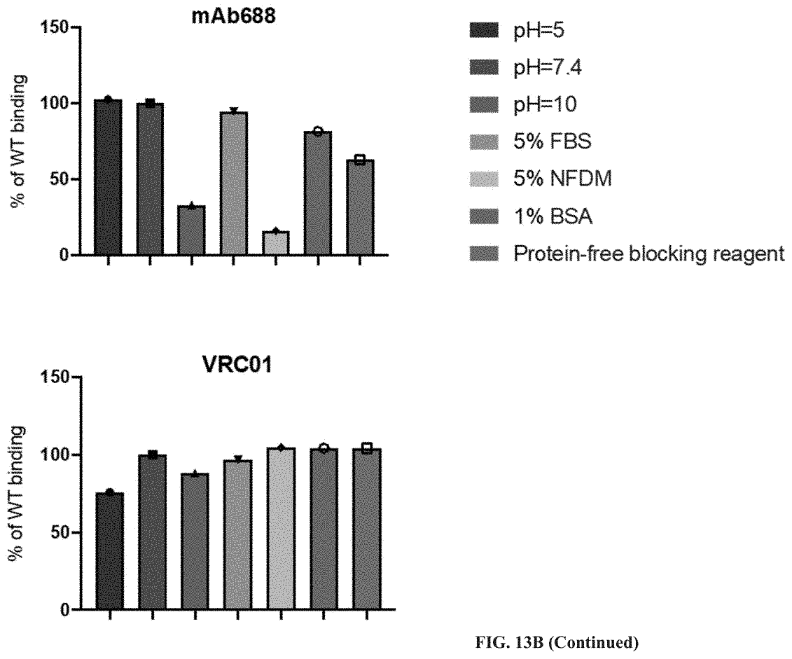

Somatic hypermutation establishes and enhances cross-reactivity. The effect of affinity maturation on the development of HIV-1/HCV cross-reactivity was interrogated. High affinity HIV-1-specific antibody responses often require the accumulation of mutations through multiple rounds of somatic hypermutation over the course of chronic infection. Binding of germline-reverted IgG antibody mutants to both HIV-1 and HCV envelope proteins was therefore assessed (FIGS. 12A and 12B, FIG. 13A). These mutants lack all acquired mutations but bear the CDR3 sequences of the mature antibody. When somatic mutations were removed from mAb688, this antibody was no longer capable of recognizing either HIV-1 or HCV (FIG. 13A). By contrast, when mAb180 and mAb692 were germline-reverted, they retained binding to HCV envelope protein, and demonstrated distinct HIV-1 envelope reactivities (FIGS. 12A and 12B). Together, these data indicate that affinity maturation is essential for both establishing and developing HIV-1/HCV cross-reactive antibody binding.

Finally, to trace the early development of HIV-1/HCV cross-reactivity, this study performed deep, unpaired BCR sequencing of donor VC10014 approximately 0.59 years post co-infection (˜3 years before the sample used for LIBRA-seq). This dataset identified multiple heavy and light chain sequences clonally related to both mAb180 and mAb692. The effect of these early acquired mutations on HIV-1/HCV antigen cross-reactivity was then defined, by expressing pairwise combinations of heavy and light chain sequences as recombinant antibodies (FIGS. 12C, 12D). Overall, distinct binding patterns were observed when various mutation-containing heavy and light chain sequences were combined (FIGS. 12C and 12D). Notably, HCV envelope recognition by both mAb180 and mAb692 was significantly more tolerant of variable somatic hypermutation than HIV-1 envelope recognition (FIGS. 12C and 12D). Together, these results indicate that different acquired-mutations or regions of the mAb180 and mAb692 paratope are essential for recognition of HCV vs. HIV-1 envelope glycoprotein.

Although antibodies are generally utilized for their incredible specificity, flexibility in the antigen binding site can provide a unique advantage in the fight against highly mutable pathogens such as HIV-1 and HCV. This is exemplified by the discovery of broadly reactive or broadly neutralizing antibodies (bNAbs) and their documented utility as prophylactic therapeutics and vaccine design scaffolds. In this study, the concept of broadly reactive antibodies is expanded by discovering the first HIV-1/HCV cross-functional antibodies. Using the LIBRA-seq technology, five genetically unique, class-switched, paired heavy-light chain sequences were identified that are positive for at least one HIV-1 and one HCV envelope glycoprotein, and then confirmed this unique antigen cross-reactivity by expression as recombinant human antibodies. All five antibodies were capable of potentiating anti-HIV-1 and anti-HCV functions. Moreover, it was found that when native antibody isotype (either IgG3-mAb688 or IgA-mAb803/mAbKP1-8) was switched to IgG1, antibody binding was reduced or ablated (FIG. 13), indicating that antigen recognition by these antibodies can be influenced by factors outside of direct antibody-epitope interactions. Further, it was discovered that somatic hypermutation aided in the development of HIV-1/HCV cross-reactivity, and mapping studies indicate that differential mutations enhance recognition of each viral antigen.

Previous studies have noted roles for poly- and auto-reactive antibodies in immune responses against highly diverse viruses, most notably observing common cross-reactivity between HIV-1 gp41-specific antibodies with host and microbiome antigens. However, these antibodies are often IgM, non-functional, or difficult to elicit by vaccination due to immune tolerance mechanisms. Notably, three of the five antibodies described in this study did not show evidence for polyreactivity. While the other two, mAb180 and mAb692, showed reactivity with host antigens, they nevertheless exhibited anti-viral functions against both HIV-1 and HCV, indicating such antibodies can still contribute to cross-viral clearance. In addition, all five antibodies regardless of autoreactivity showed exceptional HCV neutralization, inhibiting infection with 19/19 genotype 1 strains. Further, all five antibodies displayed exceptional HCV neutralization breadth, with four of the five antibodies capable of neutralizing viral strains from HCV genotypes 1-3. This is particularly striking as genotypes 1-3 account for >95% of all HCV infections in the United States. Beyond neutralization, these antibodies may still impede or reduce infection by Fc-mediated effector functions or by targeting glycan structures outside the receptor binding site to prevent viral interaction with host cell lectin receptors (DC-SIGN and L-SIGN both interact with HIV-1 and HCV).

Recent reports have outlined a class of glycan-reactive antibodies capable of recognizing, and in particular cases potentiate effector functions against, HIV-1, coronavirus, and influenza antigens, similar to the exceptionally broad antibody mAb688 isolated in the studies. Although some antibodies in this category displayed promiscuous polyreactivity against arbitrary unrelated antigens, w autoreactive binding for mAb688 was not observed. Glycan-reactive antibodies that avoid self-recognition represent a general immune defense mechanism for effectively counteracting viral infections. In addition to this glycan-reactive “class” of cross-reactive antibodies, the discovery of multiple HIV-1/HCV cross-reactive antibodies targeting diverse epitope determinants shows that there are multiple mechanisms that result in antibody cross-reactivity against unrelated antigens. These findings provide unexpected insights into the dynamic and flexible nature of the human antibody response. In the face of highly mutable threats, antibodies that can tolerate sequence variability, without triggering auto- or poly-reactivity, provide a selective advantage. Such antibodies can aid cross-reactive vaccine design or serve as therapeutics themselves for both HIV-1 and HCV, as well as emerging threats such as SARS-CoV-2 and other infectious diseases.

### Example 3. Materials and Methods

Donor information. Donor VC10014 was identified and enrolled in the Vanderbilt cohort (VC) and samples isolated after informed consent. VC10014 was recruited with CD4+ T-cell counts of >250/μl without antiretroviral therapy and with no AIDS-defining illness during the period of observation. The sample used for this study was collected on Mar. 21, 2006, >3 years post onset of co-infection.

Purification of antigens. Plasmids encoding the following genes were transfected in FreeStyle293F (Thermo Fisher Scientific) cells via polyethyleneimine transfection; HIV-1/BG505.664.SOSIP, B41.664.SOSIP, ConC gp120, A244 gp120, HCV/H77 E1E2, H77 E2c, JFH-1 E2c. All antigens contained an AviTag sequence for subsequent biotinylation. Antigens were purified by Galanthus nivalis (GNA, Snowdrop) lectin affinity chromatography (Vector Labs), and further purified by gel filtration with Superdex200 Increase column (Cytiva). Fractions corresponding to correctly folded protein were collected and biotinylated using BirA (Avidity). Biotinylated HIV-1 antigens were fluorescently labeled by incubation with Streptavidin-AF568 (Invitrogen), and biotinylated HCV antigens fluorescently labeled by incubation with Streptavidin-AF647 (Invitrogen).

The following reagents were obtained through the NIH HIV Reagent Program, Division of AIDS, NIAID, NIH: Human Immunodeficiency Virus Type 1 MN gp41 Protein, Recombinant from Escherichia coli, ARP-12027, contributed by DAIDS/NIAID; produced by ImmunoDX, LLC; Human Immunodeficiency Virus 1 (HIV-1) gp120 Recombinant Protein (B.9021 D11gp120), ARP-12571, contributed by Drs. Barton F. Haynes and Hua-Xin Liao. The following antigens were acquired from Sino Biological: Hepatitis C virus Envelope Glycoprotein E1/HCV-E1 (subtype 1b, strain HC-J4) Protein (His Tag); Human coronavirus (HCoV-229E) Spike Protein (S1+S2 ECD, His Tag); Human coronavirus (HCoV-NL63) Spike Protein (S1+S2 ECD, His Tag); SARS-CoV-2 (2019-nCoV) Spike S1+S2 ECD-His Recombinant Protein; SARS-CoV Spike S1+S2 ECD-His Recombinant Protein; Human coronavirus HKU1 (isolate N5) (HCoV-HKU1) Spike Protein (S1+S2 ECD, His Tag); MERS-CoV Spike Protein (S1+S2 ECD, aa 1-1297, His Tag). The following reagent was obtained through BEI Resources, NIAID, NIH: H1 Hemagglutinin (HA) Protein with C-Terminal Histidine Tag from Influenza Virus, A/New Caledonia/20/1999 (H1N1), Recombinant from Baculovirus, NR-48873; F Protein with C-Terminal Histidine Tag from Respiratory Syncytial Virus, B1, Recombinant from Baculovirus, NR-31097.

DNA-barcoding of antigens. The study used oligos that possess 15 bp antigen barcode, a sequence capable of annealing to the template switch oligo that is part of the 10× bead-delivered oligos and contain truncated TruSeq small RNA read 1 sequences in the following structure: 5′-CCTTGGCACCCGAGAATTCCANNNNNNNNNNNNNCCCATATAAGA*A*A-3′ (SEQ ID NO: 13156), where Ns represent the antigen barcode (Integrated DNA Technologies), and, and * represents a phosphorothioate bond. For each antigen, a unique DNA barcode was directly conjugated to the antigen itself. In particular, 5′amino-oligonucleotides were conjugated directly to each antigen using the Solulink Protein-Oligonucleotide Conjugation Kit (Vector Labs) according to manufacturer's instructions. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed together. This causes a stable bond to form between the protein and the oligonucleotide. The concentration of the antigen-oligo conjugates was determined by a BCA assay (Pierce), and the HyNic molar substitution ratio of the antigen-oligo conjugates was analyzed using the NanoDrop according to the Solulink protocol guidelines. AKTA FPLC (Cytiva) was used to remove excess oligonucleotide from the protein-oligo conjugates, which were also verified using SDS-PAGE with a silver stain (Pierce). Antigen-oligo conjugates were also used in flow cytometry titration experiments.

Antigen-specific B cell sorting. Antigen-specific B cells were sorted from donor PBMCs by fluorescence-activated cell sorting. Briefly, frozen cells were quickly thawed at 37° C., and washed 3× with DPBS without Ca2+ or Mg+ (Gibco) supplemented with 1% BSA (Sigma) (DPBS-BSA) before counting. Cells were resuspended in DPBS-BSA and stained with antibodies against cell markers including viability dye (Ghost Red 780) (Tonbo Biosciences), CD14-APC-Cy7 (BD Biosciences), IgM-APC-Cy7 (BD Biosciences), CD3-FITC (BD Biosciences), CD19-BV711 (BD Biosciences), and IgG-PE-Cy5 (BD Biosciences). Additionally, fluorescently-labeled antigen-oligo conjugates were added to the stain. After staining in the dark for 20 minutes at room temperature, cells were washed three times with DPBS-BSA. Live, CD14−, IgM−, CD3−, CD19+, Antigen+ cells were sorted using a FACSAria III flow sorter ((BD Biosciences) and to the Vanderbilt Technologies for Advanced Genomics (VANTAGE) sequencing core at an appropriate target concentration for 10× Genomics library preparation and subsequent sequencing.

Sample preparation, library preparation, and sequencing. Single-cell suspensions were loaded onto the Chromium Controller microfluidics device (10× Genomics) and processed using the B-cell Single Cell V(D)J solution according to manufacturer's indications for a target capture of 10,000 B cells per ⅛ 10× cassette, with minor modifications in order to intercept, amplify and purify the antigen barcode libraries as previously described.

Sequence processing and bioinformatic analysis. A modified version of the previously described pipeline was used. It used paired-end FASTQ files of oligo libraries as input, process and annotate reads for cell barcode, UMI, and antigen barcode, and generate a cell barcode—antigen barcode UMI count matrix. BCR contigs were processed using Cell Ranger (10× Genomics) using GRCh38 as reference. Antigen barcode libraries were also processed using Cell Ranger (10× Genomics). The overlapping cell barcodes between the two libraries were used as the basis of the subsequent analysis. Cell barcodes that had only non-functional heavy chain sequences as well as cells with multiple functional heavy chain sequences and/or multiple functional light chain sequences were removed, reasoning that these can be multiplets. Additionally, the BCR contigs (filtered_contigs.fasta file output by Cell Ranger, 10× Genomics) were aligned to IMGT reference genes using HighV-Quest38. The output of HighV-Quest was parsed using ChangeO and merged with an antigen barcode UMI count matrix. Finally, the LIBRA-seq score for each antigen in the library for every cell was determined as previously described.

Antibody purification. For each antibody, variable genes were inserted into custom plasmids encoding the native (IgG1, IgG3, or IgA2) constant region for the heavy chain as well as respective lambda or kappa light chains (pTwist CMV BetaGlobin WPRE Neo vector, Twist Bioscience). Antibodies were expressed in Expi293F mammalian cells (Thermo Fisher Scientific) by co-transfecting heavy chain and light chain expressing plasmids using polyethylenimine transfection reagent. Antibodies were purified from filtered cell supernatant by Protein A affinity chromatography, and stored in PBS, pH=7.4 unless otherwise noted. The following reagents were obtained through the NIH HIV Reagent Program, Division of AIDS, NIAID, NIH: Anti-Human Immunodeficiency Virus (HIV)-1 gp41 Monoclonal Antibody (2F5), ARP-1475, contributed by DAIDS/NIAID; Monoclonal Anti-Human Immunodeficiency Virus (HIV)-1 gp120 Protein (VRC01), ARP-12033, contributed by Dr. John Mascola; Anti-Human Immunodeficiency Virus 1 (HIV-1) gp41 Monoclonal (5F3), ARP-6882, contributed by Polymun Scientific.

ELISA. To assess antibody binding, soluble protein was plated on Immulon 2HB plates (Thermo Fisher Scientific) at 2 μg/ml overnight at 4° C. In cases where capture ELISA was used, plates were pre-incubated for 2 hours at room temperature (RT) with 5 μg/ml GNA lectin (Sigma) or 2 μg/ml anti-AviTag (Genscript) and washed 3× with PBS+0.05% Tween-20 (PBS-T) before antigen plating overnight. Between each of the subsequent incubation steps, plates were washed 3× with PBS-T. Non-specific binding was blocked by incubation with 5% fetal bovine serum (FBS) (Gibco) diluted in PBS-T for 1 hour at RT. Primary monoclonal antibodies were diluted in 5% FBS-PBST starting at 20 μg/ml with a serial 1:5 dilution (unless otherwise specified) and then added to the plate for 1 hour at RT. Secondary antibody, either goat anti-human IgG (Southern Biotech) or goat anti-human IgA (Invitrogen), was diluted 1:10,000 in 5% FBS diluted in PBS-T and added for 1 hour at RT. Reaction was developed by 10 minute incubation with One Step Ultra-TMB (Thermo Fisher Scientific) and stopped with 1N sulfuric acid. Plate absorbances were read at 450 nm (Biotek). Data are represented as mean±SEM for one ELISA experiment performed in duplicate. ELISA experiments were repeated with at least 2 different antibody preparation aliquots. The area under the curve (AUC) was calculated using GraphPad Prism 8.0.0.

Competition ELISA. Competition ELISA experiments were performed as above with minor modifications. After coating with antigen and blocking, non-biotinylated competitor antibody was added to each well at 10 μg/ml and incubated at RT for 1 hour. After washing, biotinylated antibody (final concentration of 1 μg/ml) was added and incubated for 1 hour at RT. After washing three times with PBS-T, streptavidin-HRP (Thermo Fisher Scientific) was added at 1:10,000 dilution in 5% FBS in PBS-T and incubated for 1 hour at room temperature. Plates were washed and substrate and sulfuric acid were added as described above.

Mannose-competition ELISA. Mannose competition ELISAs were performed as described above with minor modifications. After antigen coating and washing, nonspecific binding was blocked by incubation with 5% FBS diluted in PBS for 1 hour at RT. Primary antibodies were diluted in 5% FBS-PBST+/−1M D-(+)-Mannose (Sigma) starting at 10 μg/ml with a serial 1:5 dilution and then added to the plate for 1 hour at RT. After washing, antibody binding was detected with goat anti-human IgG-HRP (Southern Biotech) and added at 1:10,000 dilution in 5% FBS in PBS-T to the plates. After 1 hour incubation, plates were washed and substrate and sulfuric acid were added as described above. Data shown is representative of experiments performed in duplicate with at least 2 different antibody preparations.

HCV epitope knockout mutant binding screen. E2 mutant screening ELISAs were performed as described above with a few modifications. Plasmids encoding the gene for JFH-1 E2 containing the following mutations, as well as wild type, were transfected in Expi293F (Thermo Fisher Scientific) cells via polyethyleneimine transfection (Epitope I:L413G, N415A, N417A, W420A, N423A; Epitope II: F437A, N448A; Domain B: G523A, T526A, Y527A, W529A, N532G, T534A, D535A; Domain C: N540A, N576A; Domain D: W616A, R639A, N645A). After 4 days of expression, transfections were spun down and filtered. Immulon 2HB (Thermo Fisher Scientific) ELISA plates were pre-incubated with 2 μg/ml mouse anti-AviTag (Genscript) for 2 hours at RT, and washed 3× with PBS-T, before being coated with the above filtered cell supernatants overnight at 4 C. After antigen coating, ELISAs were performed as described above.

Negative stain grid preparation. For screening and imaging of negatively stained (NS) HIV-1 gp140 in complex with Fab 180/692, ˜3 μl of the complex after SEC at concentrations of 10 to 15 μl g/ml were applied to glow-discharged grid with continuous carbon film on 400 square mesh copper EM grids (Electron Microscopy Sciences). The grids were stained with 0.75% Uranyl formate (UF).

Screening, data collection, and image processing. NS grids were screened on an FEI Morgagni (Thermo Fisher Scientific) microscope operating at 100 kV with AMT 1 k×1 k CCD camera to verify sample and grid quality. Data collection from NS grids were done on FEI TF20 (Thermo Fisher Scientific) operate at 200 kV with US4000 4 k×4 k CCD camera (Gatan) and controlled by SerialEM. The data set was collected at nominal mag of 50K× with A/pix of 2.18 with defocus range of 1.4-1.8 and a total dose of ˜30.0 e/A2.

Image processing was performed using the CryoSPARC software package. The data set was imported, CTF estimated, and particles were picked. The particles were extracted with box size of 256×256 pixels and 2D classification was performed to generated clean homogeneous classes.

TZM-bl HIV-1 neutralization. Antibody neutralization was assessed using the TZM-bl assay. This standardized assay measures antibody-mediated inhibition of infection of JC53BL-13 cells (also known as TZM-bl cells) by molecularly cloned Env-pseudoviruses. Viruses that are highly sensitive to neutralization (Tier 1) and/or those representing circulating strains that are moderately sensitive (Tier 2) were included, plus additional viruses, including a subset of the antigens used for LIBRA-seq. Murine leukemia virus (MLV) was included as an HIV-specificity control and VRC01 was used as a positive control. Results are presented as the concentration of monoclonal antibody (in μg/ml) required to inhibit 50% of virus infection (IC₅₀).

Antibody-dependent cellular phagocytosis (ADCP), and antibody-dependent cellular cytotoxicity (ADCC). The THP-1 phagocytosis assay was performed as previously described using 1 μM neutravidin beads (Molecular Probes Inc) coated with antigen. Monoclonal IgG samples were titrated and tested at a final concentration of 100 μg/ml. Additionally monoclonal antibodies were tested starting at 100 μg/ml with 5-fold dilutions. Phagocytic scores were calculated as the geometric mean fluorescent intensity (MFI) of the beads that have been taken up multiplied by the percentage bead uptake. This, as well as all other flow cytometry work was completed on a FACSAria II (BD Biosciences). Pooled IgG from HIV-positive donors from the NIH AIDS Reagent programme (HIVIG) was used in all assays to normalize for plate to plate variation while samples from 10 Clade C-infected individuals was used as a positive control for all assays. Palivizumab (MedImmune) was used as negative control.

HCV pseudoparticle (HCV_{PP}) neutralization. A panel of 19 HCVpps were produced by lipofectamine-mediated transfection of HCV E1E2 plasmid, pNL4-3.Luc.R-E-plasmid containing the env-defective HIV proviral genome (NIH AIDS Reagent Program), and pAdVantage (Promega) into HEK293T cells. Mock pseudoparticles, generated with pNL4-3.Luc.R-E- and pAdVantage and without E1E2 plasmid, were used as a negative control for each transfection. For HCVpp testing, 8,000 Hep3B cells per well were plated in 96-well solid white flat bottom polystyrene TC-treated microplates (Corning) and incubated overnight at 37° C. For infectivity testing, HCVpp were incubated on Hep3B target cells for 5 hours. Following this incubation, medium was changed to 100 μL of phenol-free Hep3B media and incubated for 72 hours at 37° C. Infectivity was quantified using a luciferase assay as described below. All HCVpp used in neutralization assays produced RLU values at least 10-fold above background entry by mock pseudoparticles. For antibody breadth testing, HCVpp were incubated for 1 hour with mAb at 100 μg/mL and then added in duplicate to Hep3B target cells for 5 hours. Following this incubation, medium was changed to 100 μL of phenol-free Hep3B media and incubated for 72 hours at 37° C. Infectivity was quantified using a luciferase assay as described below. All HCVpp used in neutralization assays produced RLU values at least 10-fold above background entry by mock pseudoparticles. For antibody potency testing, antibodies were serially diluted five-fold, starting at a concentration of 100 μg/mL and ending at 2.56×10⁻⁴ (leaving the last well as PBS only), and incubated with HCVpp for one hour at 37° C. before the addition to HEP3B target cells in duplicate. Following this incubation, medium was changed to 100 μL of phenol-free Hep3B media and incubated for 72 hours at 37° C. After incubation (for either breadth or potency testing), media was removed from the cells, 45 μL of 1× cell culture lysis reagent (Promega) was added to each well and left to incubate for 5 minutes. The luciferase assay was measured in relative light units (RLUs) in Berthold Luminometer (Berthold Technologies Centro LB960). The percentage of neutralization for the antibody breadth was calculated as [1−(RLU_{mAb}/RLU_{IgG})]×100]. The percentage of neutralization for the dilution curves and was calculated as [1-(RLU_{mAb}/RLU_{PBS})]×100. HEPC74 and Human IgG were run as controls.

Influenza A hemagglutination inhibition (HAI). The hemagglutination inhibition (HAI) assay was used to assess the ability of mAb688 to inhibit agglutination of erythrocytes. The HAI assay was performed similarly to previously described protocols adapted from the World Health Organization (WHO) laboratory influenza surveillance manual. In brief, mAb688 (expressed as IgG1 or IgG3) was diluted in a series of 2-fold serial dilutions in v-bottom microtiter plates (Greiner Bio-One) starting from 20 μg/ml. An equal volume of A/Brisbane/02/2018 (CA/09 pdm-like H1N1) or A/Hong Kong/4801/2014 (H3N2) virus, adjusted to ˜8 hemagglutination units per 50 was added to each well. The plates were covered and incubated at room temperature for 20 min, and then a 0.8% solution of turkey (for H1N1) or guinea pig (for H3N2) erythrocytes (Lampire Biologicals) in PBS was added. Erythrocytes were stored at 4° C. and used within 72 h of preparation. The plates were mixed by agitation and covered, and the erythrocytes were settled for 30 min at room temperature. The HAI titer was determined by the reciprocal dilution of the last well that contained nonagglutinated erythrocytes. Positive and negative controls were included for each plate.

Focus reduction assay. Madin-Darby canine kidney (MDCK) cells stably-transfected with cDNA encoding human 2,6-sialtransferase (SIAT1) MDCK-SIAT1 (provided by Center for Disease Control and Prevention) were maintained in DMEM (Corning) supplemented with penicillin-streptomycin, BSA fraction V 7.5% solution (Thermo Fisher Scientific), 25 mM HEPES buffer, 10% heat-inactivated FBS and 1 mg/ml of geneticin (G418 sulfate; Thermo Fisher Scientific).

The focus reduction assay (FRA) was performed similarly to previously described protocols. In brief, MDCK-SIAT1 cells were seeded at a density of 2.5-3×10⁵ cells/ml in a 96-well plate (Greiner Bio-One) the day before the assay was run. The following day, the cell monolayers were rinsed with 0.01 M PBS (pH 7.2) (Thermo Fisher Scientific), followed by the addition of 2-fold serially diluted mAb688 (expressed as IgG1 or IgG3) at 50 μl per well starting with 20 μg/ml dilution in virus growth medium, termed VGM-T (DMEM containing 0.1% BSA, penicillin-streptomycin, and 1 m/ml L-(tosylamido-2-phenyl) ethyl chloromethyl ketone (TPCK)-treated trypsin [Sigma, St. Louis, Mo., USA]). Afterwards, 50 μl of virus (A/California/07/2009 [pdm H1N1], A/Texas/50/2012 [H3N2] or B/Massachusetts/02/2012 [influenza B virus]) standardized to 1.2×10⁴ focus forming units (FFU) per milliliter, and corresponding to 600 FFU per 50 was added to each well, including control wells. Following a 2 h incubation period at 37° C. with 5% CO₂, the cells in each well were then overlaid with 100 μl of equal volumes of 1.2% Avicel RC/CL (Type RC581 NF; FMC Health and Nutrition, Philadelphia, Pa.) in 2×MEM (Thermo Fisher Scientific) containing 1 μg/ml TPCK-treated trypsin, 0.1% BSA, and antibiotics. Plates were incubated for 18-22 h at 37° C., 5% CO₂. The overlays were then removed from each well and the monolayer was washed once with PBS to remove any residual Avicel. The plates were fixed with ice-cold 4% formalin in PBS for 30 min at 4° C., followed by a PBS wash and permeabilization using 0.5% Triton X-100 in PBS/glycine at room temperature for 20 min. Plates were washed three times with PBS supplemented with 0.1% Tween 20 (PBST) and incubated for 1 h with a mAb against influenza A nucleoprotein (provided by the International Reagent Resource (IRR), Influenza Division, WHO Collaborating Center for Surveillance, Epidemiology and Control of Influenza, Centers for Disease Control and Prevention) in ELISA buffer (PBS containing 10% horse serum and 0.1% Tween 80 [Thermo Fisher Scientific]). Following washing three time with PBST, the cells were incubated with goat anti-mouse peroxidase-labeled IgG (SeraCare) in ELISA buffer for 1 h at room temperature. Plates were washed three times with PBST, and infectious foci (spots) were visualized using TrueBlue substrate (SeraCare) containing 0.03% H₂O₂ incubated at room temperature for 10-15 min. The reaction was stopped by washing five times with distilled water. Plates were dried and foci were enumerated using an ImmunoSpot S6 ULTIMATE reader with ImmunoSpot 7.0.28.5 software (Cellular Technology Limited). The FRA titer was reported as the reciprocal of the highest dilution of serum corresponding to 50% foci reduction compared with the virus control minus the cell control.

SARS-CoV-2 pseudoparticle neutralization. To assess neutralizing activity against SARS-CoV-2 strain 2019 n-CoV/USA WA1/2020 (obtained from the Centers for Disease Control and Prevention, a gift from N. Thornburg), the high-throughput RTCA assay and xCelligence RTCA HT Analyzer (ACEA Biosciences) was used as described previously. After obtaining a background reading of a 384-well E-plate, 6,000 Vero-furin cells were seeded per well. Sensograms were visualized using RTCA HT software version 1.0.1 (ACEA Biosciences). One day later, equal volumes of virus were added to antibody samples and incubated for 1 ch at 37° C. in 5% CO₂. mAbs were tested in triplicate with a single (1:20) dilution. Virus-mAb mixtures were then added to Vero-furin cells in 384-well E-plates. Controls were included that had Vero-furin cells with virus only (no mAb) and media only (no virus or mAb). E-plates were read every 8-12 h for 72 h to monitor virus neutralization. At 32 h after virus-mAb mixtures were added to the E-plates, cell index values of antibody samples were compared to those of virus only and media only to determine presence of neutralization.

Uropathogenic E. coli hemagglutination and adherence inhibition. Hemagglutination assays were performed as described previously. Bacterial cultures were grown statically at 37° C. for 24 hours in Lysogeny broth (LB), subcultured intro fresh LB, and grown another 24 hours. Cultures were normalized to optical density (600 nm) of 1.0 in PBS, concentrated 10×, and resuspended in PBS or PBS containing 4% mannose (to competitively inhibit the type 1 pili), 20 μg/mL mAb688, or 20 μg/mL isotype control. Bacteria were added to a 96 well plate and diluted in two-fold increments. Next, guinea pig erythrocytes (Innovative Research, Inc.) were washed and suspended in PBS or PBS containing 4% mannose, 20 μg/mL mAb688, or 20 μg/mL isotype control. Erythrocytes were added to the diluted bacterial culture and incubated statically overnight at 4° C. Hemagglutination titer was determined by measuring the lowest dilution that visibly inhibited hemagglutination. Data are representative of three biological replicates performed in technical duplicate.

Autoreactivity. Monoclonal antibody reactivity to nine autoantigens (SSA/Ro, SS-B/La, Sm, ribonucleoprotein (RNP), Scl 70, Jo-1, dsDNA, centromere B, and histone) was measured using the AtheNA Multi-Lyte® ANA-II Plus test kit (Zeus scientific, Inc.). Antibodies were incubated with AtheNA beads for 30 min at concentrations of 50, 25, 12.5 and 6.25 μg/mL. Beads were washed, incubated with secondary and read on the Luminex platform as specified in the kit protocol. Data were analyzed using AtheNA software. Positive (+) specimens received a score >120, and negative (−) specimens received a score <100. Samples between 100-120 were considered indeterminate

HEp-2 cell binding. Antibody binding to whole (un-permeabilized) un-infected HEp-2 cells was measured by flow cytometry. Briefly, HEp-2 cells were collected and washed 3× with DPBS-BSA before counting. ˜1 million cells/condition were stained with a final concentration of 100 μg/ml, 10 μg/ml, or 1 μg/ml antibody diluted in DPBS-BSA for 20 minutes at 4° C. Cells were then washed 3× with DPBS-BSA and stained with either goat anti-human IgG labeled with PE (Southern Biotech) or goat anti-human IgA (Southern Biotech) labeled with PE diluted 1:1000 in DPBS-BSA for 20 minutes at 4 C. Cells were washed a final time and fluorescence acquired on a 4-Laser Fortessa (BD Biosciences). FCS files were analyzed and figures generated using CytoBank. Data shown is representative of at least 2 separate experiments with different antibody preparations. The following reagent was obtained through the NIH HIV Reagent Program, Division of AIDS, NIAID, NIH: Anti-Human Immunodeficiency Virus (HIV)-1 gp41 Monoclonal Antibody (4E10), ARP-10091, contributed by DAIDS/NIAID.

TABLE 1
LIBRA-seq Identifies Multiple Unique HIV/HCV
Cross-reactive Antibodies.
Sequences in Table:
CARSEKRVTMTRKIKGRWFGPW (SEQ ID NO: 1822);
CARVAPPGVVNNKWFDIW (SEQ ID NO: 1821);
CAAGLWSGDLSRPRYSDSW (SEQ ID NO: 1817);
CAKGLTTESRLEFW (SEQ ID NO: 1818);
CVSSWGPESPYYFDYW (SEQ ID NO: 1819);
CAREYCTGGDCHFFLDYW (SEQ ID NO: 1820);
CQVWDSSSEHVVF (SEQ ID NO: 1888);
CCLYAGSYSWVF (SEQ ID NO: 1887);
CMQPLQLPDTF (SEQ ID NO: 1883);
CQQSYNVPTF (SEQ ID NO: 1884);
CHQSSSLPFTF (SEQ ID NO: 1885);
CQHFYSSPPTF(SEQ ID NO: 1886).

| mAb | IGHV | IGHJ | % | CDR | IGLV | IGLJ | % | CDR |
|---|---|---|---|---|---|---|---|---|
| name | gene | gene | SHM | 3 | gene | gene | SHV | 3 |
| mAb180 | IGHV3- | IGHJ5 | 0.8877 | CARS | IGLV3- | IGLJ2 | 0.9283 | CQVW |
| | 48 | | | EKRV | 21 | | | DSSS |
| | | | | TMTR | | | | EHVV |
| | | | | KIKG | | | | F |
| | | | | RWFG | | | | |
| | | | | PW | | | | |
| mAb692 | IGHV1- | IGHJS | 0.9132 | CARV | IGLV2- | IGLJ3 | 0.9028 | CCLY |
| | 46 | | | APPG | 11 | | | AGSY |
| | | | | WNNK | | | | SWVF |
| | | | | SVFE | | | | |
| | | | | MW | | | | |
| mAb504 | IGHV3- | 1GHJ4 | 0.8741 | CAAG | IGKV2- | IGKJ4 | 0.9558 | CMQP |
| | 15 | | | LWSG | 28 | | | LQLP |
| | | | | DLSR | | | | DTF |
| | | | | PRYS | | | | |
| | | | | DSW | | | | |
| mAb688 | IGHV3- | IGHJ5 | 0.875 | CAKG | IGKV1- | IGKJ1 | 0.8674 | CQQS |
| | 23 | | | LTTE | 39 | | | YNVP |
| | | | | SRLE | | | | TF |
| | | | | FW | | | | |
| mAb803 | IGHV3- | IGHJ4 | 0.9514 | CVSS | IGKV6- | IGKJ3 | 0.9642 | CHQS |
| | 21 | | | WGPE | 21 | | | SSLP |
| | | | | SPYY | | | | FTF |
| | | | | FDYW | | | | |
| mAbKP1- | IGHV1- | IGHJ4 | 0.9028 | CARE | IGKV4- | IGKJ3 | 0.9327 | CQH |
| 8 | 8 | | | YCTG | 1 | | | FYSS |
| | | | | GDCH | | | | PPTF |
| | | | | FFLD | | | | |
| | | | | YW | | | | |

TABLE 2
Additional sequences

| | |
|---|---|
| SEQ ID NO: 1871 | TTGGCTTCT |
| SEQ ID NO: 1872 | GCTGCGTCC |
| SEQ ID NO: 1873 | TATGCTTCC |
| SEQ ID NO: 1874 | TGGGCATCT |
| SEQ ID NO: 1875 | GATGTCAAT |
| SEQ ID NO: 1876 | GATGATACC |
| SEQ ID NO: 4335 | GGGGAA |
| SEQ ID NO: 10982 | TTTGGCCA |
| SEQ ID NO: 11188 | AGACTC |
| SEQ ID NO: 11197 | AGCCTCAGA |
| SEQ ID NO: 11523 | GGTGCATCC |
| SEQ ID NO: 11524 | AAGGCGTCT |
| SEQ ID NO: 11525 | GAGGTCAGT |
| SEQ ID NO: 11526 | GGTGTTACC |
| SEQ ID NO: 11527 | GATACAAGC |
| SEQ ID NO: 11528 | GAAGACAGT |
| SEQ ID NO: 11529 | GGTTCATCT |
| SEQ ID NO: 11530 | AAGACGTCT |
| SEQ ID NO: 11531 | TGGGCATCT |
| SEQ ID NO: 11532 | AAAGACAAC |
| SEQ ID NO: 11533 | GATGCTTCC |
| SEQ ID NO: 11534 | GATGTCAGT |
| SEQ ID NO: 11535 | TGGGCTTCT |
| SEQ ID NO: 11536 | GGTAACAGG |
| SEQ ID NO: 11537 | GAGGTCAAT |
| SEQ ID NO: 11538 | GGTGAAAAC |
| SEQ ID NO: 11539 | GCAGATAAC |
| SEQ ID NO: 11540 | TGGGCATCG |
| SEQ ID NO: 11541 | TATGCTTCC |
| SEQ ID NO: 11542 | GGTATATCC |
| SEQ ID NO: 11543 | AGTGCATCC |
| SEQ ID NO: 11544 | GGTGCATCG |
| SEQ ID NO: 11545 | GATGATAGC |
| SEQ ID NO: 11546 | GGTAACAAT |
| SEQ ID NO: 11547 | GCTGCAGTC |
| SEQ ID NO: 11548 | GAGGGCAGT |
| SEQ ID NO: 11549 | GAAGTTTCC |
| SEQ ID NO: 11550 | GCTGCATCC |
| SEQ ID NO: 11551 | AAAGACGAT |
| SEQ ID NO: 11552 | AGGAATAAT |
| SEQ ID NO: 11553 | GAGGTCACT |
| SEQ ID NO: 11555 | GTTGCATCC |
| SEQ ID NO: 11556 | AAGGTTTCT |
| SEQ ID NO: 11557 | GGTATCACC |
| SEQ ID NO: 11558 | GATGCATCC |
| SEQ ID NO: 11560 | GGTAACAGC |
| SEQ ID NO: 11561 | GGTGTATCC |
| SEQ ID NO: 11562 | CGTGCATCC |
| SEQ ID NO: 11563 | GATACATCC |
| SEQ ID NO: 11564 | GTTACATCC |
| SEQ ID NO: 11565 | GCTGAATCC |
| SEQ ID NO: 11566 | GCTGCGTCC |
| SEQ ID NO: 11567 | GCTGCATAC |
| SEQ ID NO: 11568 | GATGCAACC |
| SEQ ID NO: 11569 | GGTGCCTCC |
| SEQ ID NO: 11570 | GACGCTTCC |
| SEQ ID NO: 11571 | GATGCGTCC |
| SEQ ID NO: 11572 | GATGTATCC |
| SEQ ID NO: 11573 | AAAGACACT |
| SEQ ID NO: 11574 | GATGACAGT |
| SEQ ID NO: 11575 | GATGTCACT |
| SEQ ID NO: 11576 | GGTGCATAC |
| SEQ ID NO: 11577 | GGTAAAAAC |
| SEQ ID NO: 11578 | CGGAATAAT |
| SEQ ID NO: 11579 | ACGCTTTCC |
| SEQ ID NO: 11580 | GGTGCCTCT |
| SEQ ID NO: 11581 | AAGGACACT |
| SEQ ID NO: 11582 | GAGGATAAC |
| SEQ ID NO: 11583 | AGTGCATCC |
| SEQ ID NO: 11584 | GGGAACGAC |
| SEQ ID NO: 11585 | GGTGCGTCC |
| SEQ ID NO: 11586 | AAAGACAGT |
| SEQ ID NO: 11587 | AATGACAGC |
| SEQ ID NO: 11588 | TCTGCATCC |
| SEQ ID NO: 11589 | AGGGCGTCT |
| SEQ ID NO: 11590 | TCTGATAAT |
| SEQ ID NO: 11591 | CAAGACAGT |
| SEQ ID NO: 11592 | CAGGCGTCT |
| SEQ ID NO: 11593 | GGCAACAAC |
| SEQ ID NO: 11594 | TTAACTTCT |
| SEQ ID NO: 11595 | GGTATTTCC |
| SEQ ID NO: 11596 | AAGGCATCT |
| SEQ ID NO: 11597 | CAAGATACC |
| SEQ ID NO: 11598 | GGTACATCC |
| SEQ ID NO: 11599 | TGGGCGTCT |
| SEQ ID NO: 11600 | GAGGTTTTT |
| SEQ ID NO: 11601 | AGCAATGAT |
| SEQ ID NO: 11602 | GCCGCCTCC |
| SEQ ID NO: 11603 | AGCACAAAC |
| SEQ ID NO: 11604 | AGGGCATCT |
| SEQ ID NO: 11605 | GGTGCTTCC |
| SEQ ID NO: 11606 | CATGCTTCC |
| SEQ ID NO: 11607 | GATGCATTC |
| SEQ ID NO: 11608 | AAGATTTCT |
| SEQ ID NO: 11609 | TCTACTTCC |
| SEQ ID NO: 11610 | GCCGCATCC |
| SEQ ID NO: 11611 | TTGGGATCT |
| SEQ ID NO: 11612 | GCTGCATTC |
| SEQ ID NO: 11613 | GATAAAGAC |
| SEQ ID NO: 11614 | AGGACTAAT |
| SEQ ID NO: 11615 | AGGGATTCT |
| SEQ ID NO: 11616 | AATGTCAGT |
| SEQ ID NO: 11618 | TCTACATCC |
| SEQ ID NO: 11619 | GATGCCTCC |
| SEQ ID NO: 11620 | GAGGATTAC |
| SEQ ID NO: 11621 | CAAGATAGC |
| SEQ ID NO: 11622 | CAGGCATCT |
| SEQ ID NO: 11623 | CATGATAGC |
| SEQ ID NO: 11624 | AATAATAAT |
| SEQ ID NO: 11625 | ACTGGAACC |
| SEQ ID NO: 11626 | GGAAGATAC |
| SEQ ID NO: 11627 | AATAAGGAC |
| SEQ ID NO: 11628 | GGTGCATCT |
| SEQ ID NO: 11629 | GGTAACATC |
| SEQ ID NO: 11630 | GATGATACC |
| SEQ ID NO: 11631 | CAGTCATCT |
| SEQ ID NO: 11632 | GGCAATAAC |
| SEQ ID NO: 11633 | GACAGTAAT |
| SEQ ID NO: 11634 | GGTGTGTCC |
| SEQ ID NO: 11635 | GAGGCGTCT |
| SEQ ID NO: 11636 | GAAGTCAGT |
| SEQ ID NO: 11638 | GGAGCGTCA |
| SEQ ID NO: 11639 | GGGGCCTCT |
| SEQ ID NO: 11640 | GATACAACT |
| SEQ ID NO: 11641 | GCTAACAGC |
| SEQ ID NO: 11642 | ATGGTTTCT |
| SEQ ID NO: 11643 | GACGTCATT |
| SEQ ID NO: 11644 | GATGTCAAT |
| SEQ ID NO: 11645 | GATGCTTCT |
| SEQ ID NO: 11646 | GAGGACAAC |
| SEQ ID NO: 11647 | AAAGACAGA |
| SEQ ID NO: 11648 | GCGGGCAGT |
| SEQ ID NO: 11649 | CAAGATACT |
| SEQ ID NO: 11650 | GGTAATAAT |
| SEQ ID NO: 11651 | GAAGTCAAT |
| SEQ ID NO: 11652 | GATGTCATT |
| SEQ ID NO: 11653 | AAGGCAGTT |
| SEQ ID NO: 11654 | AGCACAGAC |
| SEQ ID NO: 11655 | GAGGTTTCT |
| SEQ ID NO: 11656 | AACACAAGC |
| SEQ ID NO: 11657 | TTGGGTTCT |
| SEQ ID NO: 11658 | GCTACATCC |
| SEQ ID NO: 11659 | AGGGTTTCT |
| SEQ ID NO: 11660 | GATGACAGC |
| SEQ ID NO: 11661 | GAGGATACA |
| SEQ ID NO: 11662 | AAGGCCTCT |
| SEQ ID NO: 11663 | GGTGGGTCA |
| SEQ ID NO: 11664 | GGTGATAGC |
| SEQ ID NO: 11665 | GAGACTAAC |
| SEQ ID NO: 11667 | AGGAATGAT |
| SEQ ID NO: 11668 | GCTTCATCC |
| SEQ ID NO: 11669 | GACAATAAT |
| SEQ ID NO: 11670 | GCTGCCTCC |
| SEQ ID NO: 11671 | TGGGCCTCT |
| SEQ ID NO: 11672 | GAAGATACC |
| SEQ ID NO: 11673 | GATAGTTAT |
| SEQ ID NO: 11674 | GCTAAAAAC |
| SEQ ID NO: 11675 | AAGACATCT |
| SEQ ID NO: 11676 | AAGGCGTCC |
| SEQ ID NO: 11677 | ATGGCGTCT |
| SEQ ID NO: 11678 | GAAGCCTCC |
| SEQ ID NO: 11679 | AAAGACAAT |
| SEQ ID NO: 11680 | GGTAACCGC |
| SEQ ID NO: 11681 | GATGAATCC |
| SEQ ID NO: 11682 | GGTGAATCC |
| SEQ ID NO: 11683 | GACAATAGT |
| SEQ ID NO: 11684 | CAAGATAGT |
| SEQ ID NO: 11685 | GCTACATTC |
| SEQ ID NO: 11686 | CAAGATTCC |
| SEQ ID NO: 11687 | GAGGCATCC |
| SEQ ID NO: 11688 | AGCACACAC |
| SEQ ID NO: 11689 | GATGCAAAC |
| SEQ ID NO: 11690 | TGGTCATCT |
| SEQ ID NO: 11691 | GAGGTCAGG |
| SEQ ID NO: 11692 | GCTGCTTCC |
| SEQ ID NO: 11693 | TCTACATCT |
| SEQ ID NO: 11694 | GACGTCAAT |
| SEQ ID NO: 11695 | GATGATAGT |
| SEQ ID NO: 11696 | GATGCCTCT |
| SEQ ID NO: 11697 | GAAAATAAT |
| SEQ ID NO: 11698 | TTGGCTTCT |
| SEQ ID NO: 11699 | CAGGATTCT |
| SEQ ID NO: 11700 | GATACTTCC |
| SEQ ID NO: 11701 | GATGCTTTC |
| SEQ ID NO: 11702 | AGAATTTCT |
| SEQ ID NO: 11703 | AGTCATATT |
| SEQ ID NO: 11704 | AAGGCGTCA |
| SEQ ID NO: 11705 | GCCGTCAGT |
| SEQ ID NO: 11706 | GGTGCAACC |
| SEQ ID NO: 11707 | GAAGTCGTT |
| SEQ ID NO: 11708 | CATGCGTCT |
| SEQ ID NO: 11709 | AGTGACAGT |
| SEQ ID NO: 11710 | GGTATCTCC |
| SEQ ID NO: 11711 | AGTGCCTCC |
| SEQ ID NO: 11712 | GGGACTTCC |
| SEQ ID NO: 11713 | GATGATGAC |
| SEQ ID NO: 11714 | GACAACAAT |
| SEQ ID NO: 11715 | GACACTACT |
| SEQ ID NO: 11716 | AATGATAAT |
| SEQ ID NO: 11717 | GATGGATCC |
| SEQ ID NO: 11718 | GAAGTCTAT |
| SEQ ID NO: 11719 | GGGGCATCG |
| SEQ ID NO: 11720 | GACAATGAT |
| SEQ ID NO: 11721 | GATGCATCA |
| SEQ ID NO: 11722 | GAGGTTTCC |
| SEQ ID NO: 11723 | ACTAATAAT |
| SEQ ID NO: 11724 | GAAGTTACT |
| SEQ ID NO: 11725 | TCTGCTTCC |
| SEQ ID NO: 11726 | GCTGCATCG |
| SEQ ID NO: 11727 | GGGGTCACT |
| SEQ ID NO: 11728 | GGTAACAAC |
| SEQ ID NO: 11729 | GGTAAAAGG |
| SEQ ID NO: 11730 | ATGATTTCT |
| SEQ ID NO: 11731 | GCTGCAAGT |
| SEQ ID NO: 11733 | AGTAATGAT |
| SEQ ID NO: 11734 | GATGATCGG |
| SEQ ID NO: 11735 | TCTGCCTCC |
| SEQ ID NO: 11736 | TCGGTTTCT |
| SEQ ID NO: 11737 | GCTGCATTT |
| SEQ ID NO: 11738 | CTCCGTTCT |
| SEQ ID NO: 11739 | GATGTGTCC |
| SEQ ID NO: 11741 | GAAGCATCT |
| SEQ ID NO: 11742 | AGTACAAGC |
| SEQ ID NO: 11743 | GATGACAAA |
| SEQ ID NO: 11744 | GAGGGCACT |
| SEQ ID NO: 11745 | AGTACTGAT |
| SEQ ID NO: 11746 | GAAAGTTAT |
| SEQ ID NO: 11747 | GGTAACACC |
| SEQ ID NO: 11748 | AGAACATCC |
| SEQ ID NO: 11749 | CTTAACTCC |
| SEQ ID NO: 11750 | GCTGGTTAT |
| SEQ ID NO: 11751 | GATTCATCC |
| SEQ ID NO: 11752 | CAGGTTTTT |
| SEQ ID NO: 11753 | GGAGCAACC |
| SEQ ID NO: 11754 | AGCGATAAC |
| SEQ ID NO: 11755 | GATGATCGG |
| SEQ ID NO: 11756 | CTTAACAGC |
| SEQ ID NO: 11757 | AGTAGTAAT |
| SEQ ID NO: 11758 | GAGGTCCGT |
| SEQ ID NO: 11759 | GAAGTCCAT |
| SEQ ID NO: 11760 | AAAGACAAA |
| SEQ ID NO: 11761 | GCTGTATCC |
| SEQ ID NO: 11762 | GAGAATAAT |
| SEQ ID NO: 11763 | GCAGCATCC |
| SEQ ID NO: 11764 | GCGGGCAGA |
| SEQ ID NO: 11765 | GATGATAAA |
| SEQ ID NO: 11766 | GGTGTCTCC |
| SEQ ID NO: 13126 | LAS |
| SEQ ID NO: 13127 | AAS |
| SEQ ID NO: 13128 | YAS |
| SEQ ID NO: 13129 | WAS |
| SEQ ID NO: 13130 | DVN |
| SEQ ID NO: 13131 | DDT |

### Sequences

A large number of sequences were analyzed throughout this application. Additional sequences reviewed and analyzed include:

SEQ ID NOs: 1905-2627 which are “Barcode” sequences;

SEQ ID NOs: 2628-3258 which are “SEQUENCE VDJ.H” sequences;

SEQ ID NOs: 3259-3880 which are “JUNCTION.H” sequences;

SEQ ID NOs: 7771-8518 which are “SEQUENCE VDJ.L” sequences;

SEQ ID NOs: 8520-9189 which are “JUNCTION.L” sequences.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating a coronavirus infection in a subject in need comprising administering to the subject a therapeutically effective amount of a recombinant antibody, wherein the recombinant antibody comprises a light chain variable region (VL) that comprises a light chain complementarity determining region (CDRL)3 and a heavy chain variable region (VH) that comprises a heavy chain complementarity determining region (CDRH)3, wherein the CDRL3 comprises an amino acid sequence at least 60% identical to CQQSYNVPTF (SEQ ID NO: 1884), and wherein the CDRH3 comprises an amino acid sequence at least 60% identical to
(SEQ ID NO: 1818)
CAKGLTTESRLEFW.

2. The method of claim 1, wherein the coronavirus comprises MERS-CoV, SARS-CoV, or SARS-CoV-2.

3. The method of claim 1, wherein
CDRL1 is SEQ ID NO: 13121,
CDRL2 is SEQ ID NO: 13127,
CDRL3 is SEQ ID NO: 25 or 1884,
CDRH1 is SEQ ID NO: 13103,
CDRH2 is SEQ ID NO: 13109, and
CDRH3 is SEQ ID NO: 1818.

4. The method of claim 1, wherein the subject is co-infected by a pathogen.

5. The method of claim 4, wherein the pathogen is a virus or a bacterium.

6. The method of claim 5, wherein the virus is selected from the group consisting of influenza A, influenza B, HIV, and HCV.

7. The method of claim 5, wherein the bacterium is Escherichia coli.

8. A method of treating an influenza infection in a subject in need comprising administering to the subject a therapeutically effective amount of a recombinant antibody, wherein the recombinant antibody comprises a light chain variable region (VL) that comprises a light chain complementarity determining region (CDRL)3 and a heavy chain variable region (VH) that comprises a heavy chain complementarity determining region (CDRH)3, wherein the CDRL3 comprises an amino acid sequence at least 60% identical to CQQSYNVPTF (SEQ ID NO: 1884), and wherein the CDRH3 comprises an amino acid sequence at least 60% identical to
(SEQ ID NO: 1818)
CAKGLTTESRLEFW.

9. The method of claim 8, wherein
CDRL1 is SEQ ID NO: 13121,
CDRL2 is SEQ ID NO: 13127,
CDRL3 is SEQ ID NO: 25 or 1884,
CDRH1 is SEQ ID NO: 13103,
CDRH2 is SEQ ID NO: 13109, and
CDRH3 is SEQ ID NO: 1818.

10. The method of claim 8, wherein the influenza is influenza A or influenza B.

11. The method of claim 8, wherein the subject is co-infected by a pathogen.

12. The method of claim 11, wherein the pathogen is a virus or a bacterium.

13. The method of claim 12, wherein the virus is selected from the group consisting of MERS-CoV, SARS-CoV, SARS-CoV-2, HIV, and HCV.

14. The method of claim 12, wherein the bacterium is Escherichia coli.

15. A method of treating an Escherichia coli infection in a subject in need comprising administering to the subject a therapeutically effective amount of a recombinant antibody, wherein the recombinant antibody comprises a light chain variable region (VL) that comprises a light chain complementarity determining region (CDRL)3 and a heavy chain variable region (VH) that comprises a heavy chain complementarity determining region (CDRH)3, wherein the CDRL3 comprises an amino acid sequence at least 60% identical to CQQSYNVPTF (SEQ ID NO: 1884), and wherein the CDRH3 comprises an amino acid sequence at least 60% identical to
(SEQ ID NO: 1818)
CAKGLTTESRLEFW.

16. The method of claim 15, wherein
CDRL1 is SEQ ID NO: 13121,
CDRL2 is SEQ ID NO: 13127,
CDRL3 is SEQ ID NO: 25 or 1884,
CDRH1 is SEQ ID NO: 13103,
CDRH2 is SEQ ID NO: 13109, and
CDRH3 is SEQ ID NO: 1818.

17. The method of claim 15, wherein the subject is co-infected by a pathogen.

18. The method of claim 17, wherein the pathogen is a virus or a bacterium.

19. The method of claim 18, wherein the virus is selected from the group consisting of MERS-CoV, SARS-CoV, SARS-CoV-2, influenza A, influenza B, HIV, and HCV.
